(19) United States
(12) Patent Application Publication
(10) Pub. No.: US 2019/0184402 A1
(43) Pub. Date: Jun. 20, 2019

(54) METHODS AND SYSTEMS FOR ANALYZING NUCLEIC ACIDS

(71) Applicant: Coyote Bioscience USA Inc., Campbell, CA (US)

(72) Inventors: Jesus Ching, Saratoga, CA (US); Phillip You Fai Lee, South San Francisco, CA (US)

(21) Appl. No.: 16/049,672

(22) Filed: Jul. 30, 2018

Related U.S. Application Data
(63) Continuation of application No. PCT/US1017/017142, filed on Feb. 9, 2017.
(60) Provisional application No. 62/293,486, filed on Feb. 10, 2016.

(51) Int. Cl. B01L 7/00; G01N 35/00; C12Q 1/6844

(52) U.S. Cl. CPC B01L 7/5255; G01N 35/00584; B01L 2300/18; G01N 2035/00346; C12Q 1/6846

(57) ABSTRACT

The present disclosure provides methods and systems for analyzing nucleic acids and for conducting chemical and/or biological reactions.

### CROSS-REFERENCE

This application is a continuation of Patent Cooperation Treaty Application No. PCT/US2017/017142, filed on Feb. 9, 2017, which claims priority to U.S. Provisional Patent Application Ser. No. 62/293,486, filed Feb. 10, 2016, which applications are herein incorporated by reference in their entirety for all purposes.

### BACKGROUND

Nucleic acid amplification methods may permit selected amplification and identification of nucleic acids of interest from a complex mixture, such as a biological sample. To detect a nucleic acid in a biological sample, the biological sample is typically processed to isolate nucleic acids from other components of the biological sample and other agents that may interfere with the nucleic acid and/or amplification. Following isolation of the nucleic acid of interest from the biological sample, the nucleic acid of interest may be amplified, via, for example, nucleic acid amplification, such as a thermal cycling based approach (e.g., polymerase chain reaction (PCR)). Following amplification of the nucleic acid of interest, the products of amplification may be detected and the results of detection interpreted by an end-user. However, it has been tedious, time consuming and inefficient when multiple or numerous amplification reactions need to be performed.

Droplets have been proposed as containers to perform chemical and biochemical reactions (e.g., nucleic acid amplification) in confined volumes, and various methods have been developed to generate such droplets. However, these techniques often have problems associated with uneven droplet size and composition, relatively low throughput, and/or unable to generate monodisperse droplets.

In addition, in an appropriate reagent reaction system, nucleic acid amplifications can occur very rapidly. In fact, amplification of nucleic acid molecules in a polymerase chain reaction (PCR) can occur in one to two seconds, or even less than one second per cycle. Therefore, in many situations, the speed of PCR amplification is limited by the performance of the instrumentation (e.g. thermal cycler) rather than the biological reaction itself.

### SUMMARY

Recognized herein is the need for rapid, accurate and high throughput methods and devices for analyzing nucleic acids from complex sample types. Such methods and devices may be useful, for example, in realizing fast sample-to-answer detection and management of diseases detectable via their nucleic acid.

The present disclosure provides methods and systems for efficient amplification of nucleic acids, such as ribonucleic acid (RNA) and deoxyribonucleic acid (DNA) molecules, especially for amplifying and analyzing a large amount of different nucleic acid molecules with high throughput and/or in parallel. The present disclosure also provides methods and systems of rapid thermal cycling, e.g., in nucleic acid amplifications. Reducing the time for heating and cooling sample volumes between the necessary temperature set points can reduce the time to conduct a reaction cycle and therefore reduce the overall reaction time over multiple cycles. Furthermore, the present disclosure also provides methods and systems for achieving fast and convenient heat exchange (e.g., cooling) without the need for additional power supply.

In an aspect, the disclosure provides a system for conducting a chemical or biological reaction on a biological sample. The system comprises a sample holder that receives a solution comprising the biological sample and retains the solution during the chemical or biological reaction; and a plurality of thermal zones comprising at least a first thermal zone and a second thermal zone adjacent to the sample holder. The second thermal zone can be angularly separated from the first thermal zone along an axis of rotation of (1) the sample holder or (2) the plurality of thermal zones. The system also comprises a controller that alternately and sequentially positions the solution in each of the plurality of thermal zones through rotation of the sample holder or the plurality of thermal zones, to conduct the chemical or biological reaction. In the first thermal zone, the solution is subjected to heating or cooling at a first temperature profile, and, in the second thermal zone, the solution is subjected to heating or cooling at a second temperature profile that is different than the first temperature profile.

In some embodiments, the first thermal zone comprises a first heating or cooling unit that subjects the solution to heating or cooling at the first temperature profile. In some embodiments, the first heating or cooling unit may be one or more of an infrared (IR) heating unit, a convective heating unit, a Peltier, a resistive heating unit and a heating block. In some embodiments, the first heating or cooling unit is a cooling unit and may be one or more of a desiccant, a convective cooling unit and a cooling block.

In some embodiments, the second thermal zone comprises a second heating or cooling unit that subjects the solution to heating or cooling at the second temperature profile. In some embodiments, the second heating or cooling unit may be one or more of an infrared (IR) heating unit, a convective heating unit, a Peltier, a resistive heating unit and a heating block. In some embodiments, the second heating or cooling unit is a cooling unit selected and may be one or more of a desiccant, a convective cooling unit and a cooling block.

In some embodiments, the first thermal zone and the second thermal zone are included on a support. In some embodiments, the support is rotatable with respect to the sample holder. In some embodiments, the chemical or biological reaction comprises cycling the biological sample between at least two target temperature levels. In some embodiments, the solution undergoes heating and in the second thermal zone the solution undergoes cooling, or vice versa.

In some embodiments, the plurality of thermal zones further comprises a third thermal zone adjacent to the sample holder. The third thermal zone can be different than the first thermal zone and the second thermal zone. In some embodiments, the third thermal zone the solution is subjected to heating or cooling at a third temperature profile. In some embodiments, the third temperature profile is different than the first temperature profile and the second temperature profile. In some embodiments, the third temperature profile comprises a third target temperature. In some embodiments, the first temperature profile comprises a first target temperature. In some embodiments, the second temperature profile comprises a second target temperature that is different than the first target temperature.

In some embodiments, the solution comprises reagents necessary for the chemical or biological reaction. In some embodiments, the chemical or biological reaction is nucleic acid amplification and the reagents include one or more primers and polymerizing enzymes. In some embodiments, the nucleic acid amplification is polymerase chain reaction (PCR).

In some embodiments, the sample holder is rotatable with respect to the first thermal zone and the second thermal zone. In some embodiments, the first thermal zone and the second thermal zone are rotatable with respect to the solution.

In some embodiments, the controller comprises one or more computer processors that are individually or collectively programmed to alternately and sequentially position the solution in the first thermal zone and the second thermal zone. In some embodiments, the system also includes a detector adjacent to the sample holder. The detector can detect a signal from the solution that is indicative of the chemical or biological reaction or a product of the chemical or biological reaction on the biological sample. In some embodiments, the detector is angularly separated from the first thermal zone and the second thermal zone along the axis of rotation. In some embodiments, the controller positions the solution in sensing communication with the detector through rotation of the sample holder or the detector.

An additional aspect of the disclosure provides a method for conducting a chemical or biological reaction on a biological sample. The method comprises: (a) depositing a solution comprising the biological sample in a sample holder, where the sample holder retains the solution during the chemical or biological reaction, where the sample holder is disposed adjacent to a plurality of thermal zones comprising at least a first thermal zone and a second thermal zone, and where the second thermal zone is angularly separated from the first thermal zone along an axis of rotation of (1) the sample holder or (2) the plurality of thermal zones. The method also comprises: (b) alternately and sequentially positioning the solution in each of the plurality of thermal zones through rotation of the sample holder or the plurality of thermal zones, to conduct the chemical or biological reaction on the biological sample, where (i) in the first thermal zone the solution is subjected to heating or cooling at a first temperature profile, and (ii) in the second thermal zone the solution is subjected to heating or cooling at a second temperature profile that is different than the first temperature profile.

In some embodiments, (b) comprises positioning the solution in the first thermal zone and subsequently positioning the solution in the second thermal zone. In some embodiments, the method further comprises positioning the solution in the first thermal zone subsequent to positioning the solution in the second thermal zone. In some embodiments, the method further comprises positioning the solution in a third thermal zone of the plurality of thermal zones subsequent to positioning the solution in the second thermal zone. The third thermal zone can be different than the first thermal zone and the second thermal zone.

In some embodiments, (b) comprises positioning the solution in the first thermal zone and subsequently positioning the solution in a third thermal zone of the plurality of thermal zones that is different than the second thermal zone. In some embodiments, the method further comprises positioning the solution in the second thermal zone subsequent to positioning the solution in the third thermal zone. In some embodiments, in the first thermal zone, the solution undergoes cooling and in the second thermal zone the solution undergoes heating, or vice versa. In some embodiments, the first temperature profile comprises a first target temperature. In some embodiments, the second temperature profile comprises a second target temperature that is different than the first target temperature.

In some embodiments, the solution comprises reagents necessary for the chemical or biological reaction. In some embodiments, the chemical or biological reaction is nucleic acid amplification and the reagents include one or more primers and polymerizing enzymes. In some embodiments, the nucleic acid amplification is polymerase chain reaction (PCR).

In some embodiments, the sample holder is rotatable with respect to the first thermal zone and the second thermal zone. In some embodiments, in (b), the sample holder rotates the solution from the first thermal zone to the second thermal zone, such that the solution is in thermal communication with the second thermal zone. In some embodiments, the first thermal zone and the second thermal zone are rotatable with respect to the solution. In some embodiments, in (b), the second thermal zone is rotated and brought in thermal communication with the solution.

In some embodiments, the method further comprises positioning the solution in sensing communication with a detector adjacent to the sample holder. The detector can detect a signal from the solution that is indicative of the chemical or biological reaction or a product of the chemical or biological reaction on the biological sample. In some embodiments, the detector is angularly separated from the first thermal zone and the second thermal zone along the axis of rotation. In some embodiments, the solution is positioned in sensing communication with the detector through rotation of the sample holder or the detector.

An additional aspect of the disclosure provides an apparatus for generating at least one droplet comprising a biological sample for use in a chemical or biological reaction. The apparatus comprises a first chamber comprising a first fluid volume and at least one first fluid flow port that is in fluid communication with the first fluid volume. The first fluid volume retains an aqueous solution comprising the biological sample for use in the chemical or biological reaction. The apparatus also comprises a second chamber comprising a second fluid volume and at least one second fluid flow port that is in fluid communication with the second fluid volume. The second chamber at least partially circumscribes the first chamber, the second fluid volume retains a continuous fluid that is immiscible with the aqueous solution, and the second chamber is rotatable with respect to the first chamber, or vice versa. During use, rotation of the first chamber or the second chamber brings the first fluid flow port in alignment with the second fluid flow port to subject the aqueous solution comprising the biological sample to flow from the first fluid volume to the second fluid volume to generate the at least one droplet upon the aqueous solution contacting the continuous fluid, which at least one droplet comprises the biological sample or a portion thereof.

In some embodiments, the at least one first fluid flow port and/or the at least one second fluid flow port are dimensioned such that the at least one droplet has a predetermined characteristic size and/or shape. In some embodiments, the second chamber is rotatable with respect to the first chamber. In some embodiments, the first chamber is rotatable with respect to the second chamber. In some embodiments, the first fluid flow port is in selective alignment with the second fluid flow port upon rotation of the first chamber or the second chamber.

In some embodiments, the at least one droplet comprises a plurality of droplets. In some embodiments, each of the plurality of droplets comprises the biological sample or a portion thereof. In some embodiments, the first chamber and/or the second chamber is cylindrical. In some embodiments, the at least one first fluid flow port comprises a plurality of first fluid flow ports. In some embodiments, the plurality of first fluid flow ports are brought in alignment with the at least one second fluid flow port upon rotation of the first chamber or the second chamber. In some embodiments, the at least one second fluid flow port comprises a plurality of second fluid flow ports. In some embodiments, the plurality of second fluid flow ports are brought in alignment with the at least one first fluid flow port upon rotation of the first chamber or the second chamber.

In some embodiments, the first fluid volume is in fluid communication with a source of positive pressure that subjects the aqueous solution to flow from the first fluid volume to the second fluid volume when the first fluid flow port is aligned with the second fluid flow port, to generate the one or more droplets upon contact with the continuous fluid. In some embodiments, the source of positive pressure subjects the first chamber or the second chamber to rotation. In some embodiments, the source of positive pressure is a compressor or a plunger. In some embodiments, the source of positive pressure is a plunger that is actuatable by a user and/or by a mechanical unit to generate positive pressure. In some embodiments, the second fluid volume is in fluid communication with a source of negative pressure that subjects the aqueous solution to flow from the first fluid volume to the second fluid volume when the first fluid flow port is aligned with the second fluid flow port, to generate the one or more droplets upon contact with the continuous fluid.

In some embodiments, the continuous fluid comprises an oil, such as a fluorine-containing oil (e.g., a fluorocarbon oil). In some embodiments, the continuous fluid comprises a surfactant. In some embodiments, the second chamber fully circumscribes the first chamber. In some embodiments, the aqueous solution comprises reagents necessary for the chemical or biological reaction. In some embodiments, the chemical or biological reaction is nucleic acid amplification and the reagents include one or more primers and polymerizing enzymes. In some embodiments, the nucleic acid amplification is polymerase chain reaction (PCR).

In some embodiments, the second chamber comprises an inner partition and an outer partition circumscribing the inner partition. The inner partition and the outer partition at least partially define the second fluid volume and the inner partition may be adjacent to the first chamber. In some embodiments, the inner partition is in contact with the first chamber. In some embodiments, the apparatus also includes a fluid flow path between the first chamber and the inner partition.

In some embodiments, the aqueous solution is not subjected to flow from the first fluid volume to the second fluid volume in the absence of the first fluid flow port being in alignment with the second fluid flow port. In some embodiments, the at least one droplet has a size that is at least partially dependent on a rate of rotation of the first chamber or the second chamber.

In another aspect, the disclosure provides a method for generating at least one droplet comprising a biological sample for use in a chemical or biological reaction. The method comprises: (a) activating an apparatus comprising (1) a first chamber comprising a first fluid volume and at least one first fluid flow port that is in fluid communication with the first fluid volume, where the first fluid volume comprises an aqueous solution comprising the biological sample for use in the chemical or biological reaction; and (2) a second chamber comprising a second fluid volume and at least one second fluid flow port that is in fluid communication with the second fluid volume, where the second chamber at least partially circumscribes the first chamber, where the second fluid volume retains a continuous fluid that is immiscible with the aqueous solution, and where the second chamber is rotatable with respect to the first chamber, or vice versa. The method also comprises: (b) rotating the first chamber or the second chamber to bring the first fluid flow port in alignment with the second fluid flow port to subject the aqueous solution comprising the biological sample to flow from the first fluid volume to the second fluid volume to generate the at least one droplet upon the aqueous solution contacting the continuous fluid, which at least one droplet comprises the biological sample or a portion thereof.

In some embodiments, the activating comprises depositing the aqueous solution comprising the biological sample in the first fluid volume. In some embodiments, the at least one first fluid flow port and/or the at least one second fluid flow port are dimensioned such that the at least one droplet has a predetermined characteristic size and/or shape. In some embodiments, the rotating in (b) comprises rotating the second chamber with respect to the first chamber. In some embodiments, the rotating in (b) comprises rotating the first chamber with respect to the second chamber.

In some embodiments, the first fluid flow port is in selective alignment with the second fluid flow port upon rotation of the first chamber or the second chamber. In some embodiments, the at least one droplet comprises a plurality of droplets. In some embodiments, each of the plurality of droplets comprises the biological sample or a portion thereof. In some embodiments, the first chamber and/or the second chamber is cylindrical.

In some embodiments, the at least one first fluid flow port comprises a plurality of first fluid flow ports. In some embodiments, the plurality of first fluid flow ports are brought in alignment with the at least one second fluid flow port upon rotation of the first chamber or the second chamber. In some embodiments, the at least one second fluid flow port comprises a plurality of second fluid flow ports. In some embodiments, the plurality of second fluid flow ports are brought in alignment with the at least one first fluid flow port upon rotation of the first chamber or the second chamber.

In some embodiments, the first fluid volume is in fluid communication with a source of positive pressure that subjects the aqueous solution to flow from the first fluid volume to the second fluid volume when the first fluid flow port is aligned with the second fluid flow port, to generate the one or more droplets upon contact with the continuous fluid. In some embodiments, the source of positive pressure subjects the first chamber or the second chamber to rotation. In some embodiments, the source of positive pressure is a compressor or a plunger. A plunger may be actuatable by a user and/or actuatable by a mechanical unit to generate positive pressure. In some embodiments, the second fluid volume is in fluid communication with a source of negative pressure that subjects the aqueous solution to flow from the first fluid volume to the second fluid volume when the first fluid flow port is aligned with the second fluid flow port, to generate the one or more droplets upon contact with the continuous fluid.

In some embodiments, the continuous fluid comprises an oil, such as a fluorine-containing oil (e.g., a fluorocarbon oil). In some embodiments, the continuous fluid further comprises a surfactant. In some embodiments, the second chamber fully circumscribes the first chamber. In some embodiments, the aqueous solution comprises reagents necessary for the chemical or biological reaction. In some embodiments, the chemical or biological reaction is nucleic acid amplification and the reagents include one or more primers and polymerizing enzymes. In some embodiments, the nucleic acid amplification is polymerase chain reaction (PCR).

In some embodiments, the second chamber comprises an inner partition and an outer partition circumscribing the inner partition. The inner partition and the outer partition at least partially define the second fluid volume, and the inner partition may be adjacent to the first chamber. In some embodiments, the inner partition is in contact with the first chamber. In some embodiments, the apparatus comprises a fluid flow path between the first chamber and the inner partition. In some embodiments, the aqueous solution is not subjected to flow from the first fluid volume to the second fluid volume in the absence of the first fluid flow port being in alignment with the second fluid flow port. In some embodiments, the at least one droplet has a size that is at least partially dependent on a rate of rotation of the first chamber or the second chamber.

An additional aspect of the disclosure provides an apparatus for cooling a solution comprising a nucleic acid sample during a nucleic acid amplification reaction. The apparatus comprises: a first chamber comprising a heat transfer material having a phase transition temperature in a range of about −100° C. to 50° C.; and a second chamber comprising a substrate having a heat transfer surface. The second chamber is fluidically isolated from the first chamber and the heat transfer surface is in thermal communication with the solution comprising the nucleic acid sample during the nucleic acid amplification reaction. The apparatus also comprises a control unit that brings the second chamber in fluid communication with the first chamber in accordance with a timing that at least partially depends upon a duration of the nucleic acid amplification reaction. When the second chamber is in fluid communication with the first chamber, the heat transfer material can undergo a phase transition that draws thermal energy from the substrate along the heat transfer surface to subject the solution to cooling.

In some embodiments, the heat transfer surface is in thermal communication with the solution indirectly through at least one heat transfer medium. In some embodiments, the at least one heat transfer medium is a cooling fluid. In some embodiments, the heat transfer surface is in thermal communication with the solution directly.

In some embodiments, the apparatus further comprises a seal between the first chamber and the second chamber. The seal (i) can isolate the second chamber from the first chamber when in a closed configuration, and (ii) can bring the second chamber in fluid communication with the first chamber when in an open configuration. In some embodiments, during use, (i) the seal is actuated from the closed configuration to the open configuration to bring the first chamber in fluid communication with the second chamber, and (ii) the heat transfer material undergoes a phase transition, which phase transition draws thermal energy from the substrate along the heat transfer surface to subject the solution to cooling. In some embodiments, during use, the heat transfer material is subjected to flow from the first chamber to the second chamber to come in contact with the heat transfer surface, and upon contact with the heat transfer surface, the heat transfer material undergoes the phase transition to yield a vapor, which phase transition draws thermal energy from the substrate along the heat transfer surface to subject the solution to cooling. In some embodiments, the seal is part of a fluid flow path between the first chamber and the second chamber. In some embodiments, the seal is actuated from the closed configuration to the opening configuration by piercing. In some embodiments, the seal is part of a valve between the first chamber and the second chamber. The seal can be actuated from the closed configuration to the open configuration by opening the valve.

In some embodiments, the heat transfer material is a heat transfer liquid. In some embodiments, during use, the heat transfer liquid is subjected to flow from the first chamber to the second chamber to come in contact with the heat transfer surface. In some embodiments, the heat transfer liquid comprises water. In some embodiments, the heat transfer liquid comprises an alcohol, such as isopropyl alcohol, methanol, ethanol, propanol, butanol or pentanol. In some embodiments, the heat transfer material comprises a vapor pressure of at least 3 kPa. In some embodiments, the heat transfer material comprises a carbon backbone. In some embodiments, the carbon backbone comprises at most seven carbon atoms.

In some embodiments, the apparatus further comprises a third chamber in fluid communication with the second chamber through at least one fluid flow path between the second chamber and the third chamber. The third chamber can receive a vapor generated upon the heat transfer material undergoing the phase transition. In some embodiments, the third chamber comprises a capture material that captures the vapor. In some embodiments, the capture material comprises a hygroscopic material. In some embodiments, the capture material comprises a desiccant.

In some embodiments, the third chamber is in fluid communication with a pump that draws the vapor. In some embodiments, the third chamber is in fluid communication with a fluid flow unit that subjects the vapor to flow from the third chamber to a vapor repository. In some embodiments, the fluid flow unit comprises a fan, a compressor and/or a pump. In some embodiments, the substrate comprises an additional heat transfer surface. During use, a cooling fluid can be brought in contact with the additional heat transfer surface to subject the cooling fluid to cooling. In some embodiments, the control unit comprises one or more computer processors that are individually or collectively programmed to bring the second chamber in fluid communication with the first chamber in accordance with the timing.

An additional aspect of the disclosure provides an apparatus for cooling a solution comprising a nucleic acid sample during a nucleic acid amplification reaction. The apparatus comprises a first chamber comprising a heat transfer material; and a second chamber comprising a substrate having a heat transfer surface. The second chamber can be fluidically isolated from the first chamber and the heat transfer surface can be in thermal communication with the solution comprising the nucleic acid sample during the nucleic acid amplification reaction. The apparatus also comprises a seal between the first chamber and the second chamber, which seal (i) isolates the second chamber from the first chamber when in a closed configuration, and (ii) brings the second chamber in fluid communication with the first chamber when in an open configuration. During use, the seal is actuated from the closed configuration to the open configuration to bring the first chamber in fluid communication with the second chamber. In the open configuration, the heat transfer material undergoes a phase transition that draws thermal energy from the substrate along the heat transfer surface to subject the solution to cooling.

In some embodiments, the heat transfer surface is in thermal communication with the solution indirectly through at least one heat transfer medium. In some embodiments, the at least one heat transfer medium is a cooling fluid. In some embodiments, the heat transfer surface is in thermal communication with the solution directly. In some embodiments, during use, the heat transfer material is subjected to flow from the first chamber to the second chamber to come in contact with the heat transfer surface, and upon contact with the heat transfer surface, the heat transfer material undergoes the phase transition that draws thermal energy from the substrate along the heat transfer surface.

In some embodiments, the seal is part of a fluid flow path between the first chamber and the second chamber. In some embodiments, the seal is actuated from the closed configuration to the opening configuration by piercing. In some embodiments, the seal is part of a valve between the first chamber and the second chamber. The seal can be actuated from the closed configuration to the open configuration by opening the valve.

In some embodiments, the heat transfer material is a heat transfer liquid. In some embodiments, the apparatus further comprises a third chamber in fluid communication with the second chamber through at least one fluid flow path between the second chamber and the third chamber. The third chamber can receive a vapor generated upon the heat transfer material undergoing the phase transition. In some embodiments, the third chamber comprises a capture material that captures the vapor. The capture material can comprise a hygroscopic material and/or may comprise a desiccant. In some embodiments, the substrate comprises an additional heat transfer surface. During use, a cooling fluid can be brought in contact with the additional heat transfer surface to subject the cooling fluid to cooling.

An additional aspect of the disclosure provides a method for cooling a solution comprising a nucleic acid sample during a nucleic acid amplification reaction. The method comprises: (a) activating a heat exchange apparatus comprising (1) a first chamber comprising a heat transfer material having a phase transition temperature in a range of about −100° C. to 50° C.; and (2) a second chamber comprising a substrate having a heat transfer surface. The second chamber can be fluidically isolated from the first chamber and the heat transfer surface is in thermal communication with a solution comprising the nucleic acid sample during the nucleic acid amplification reaction. The method also comprises: (b) bringing the second chamber in fluid communication with the first chamber in accordance with a timing that at least partially depends upon a duration of the nucleic acid amplification. When the second chamber is in fluid communication with the first chamber, the heat transfer material can undergo a phase transition that draws thermal energy from the substrate along the heat transfer surface to subject the solution to cooling.

In some embodiments, the method further comprises (c) subjecting the solution to cooling using the thermal energy drawn from the substrate along the heat transfer surface. In some embodiments, the heat transfer surface is in thermal communication with the solution indirectly through at least one heat transfer medium. In some embodiments, the at least one heat transfer medium is a cooling fluid. In some embodiments, the heat transfer surface is in thermal communication with the solution directly.

In some embodiments, the apparatus comprises a seal between the first chamber and the second chamber, which seal (i) isolates the second chamber from the first chamber when in a closed configuration, and (ii) brings the second chamber in fluid communication with the first chamber when in an open configuration. In some embodiments, (b) comprises actuating the seal from the closed configuration to the open configuration to bring the first chamber in fluid communication with the second chamber. When the seal is in an open configuration, the heat transfer material undergoes a phase transition that draws thermal energy from the substrate along the heat transfer surface to subject the solution to cooling. In some embodiments, (b) comprises subjecting the heat transfer material to flow from the first chamber to the second chamber to come in contact with the heat transfer surface. Upon contact with the heat transfer surface, the heat transfer material undergoes the phase transition to yield a vapor. In some embodiments, the seal is part of a fluid flow path between the first chamber and the second chamber. In some embodiments, the seal is actuated from the closed configuration to the opening configuration by piercing. In some embodiments, the seal is part of a valve between the first chamber and the second chamber. The seal can be actuated from the closed configuration to the open configuration by opening the valve.

In some embodiments, the heat transfer material is a heat transfer liquid. In some embodiments, (b) comprises subjecting the heat transfer liquid to flow from the first chamber to the second chamber to come in contact with the heat transfer surface. In some embodiments, the heat transfer liquid comprises water. In some embodiments, the heat transfer liquid comprises an alcohol. In some embodiments, the alcohol comprises isopropyl alcohol, methanol, ethanol, propanol, butanol or pentanol. In some embodiments, the heat transfer material comprises a vapor pressure of at least 3 kPa. In some embodiments, the heat transfer material comprises a carbon backbone. In some embodiments, the carbon backbone comprises at most seven carbon atoms.

In some embodiments, the apparatus further comprises a third chamber in fluid communication with the second chamber through at least one fluid flow path between the second chamber and the third chamber. The third chamber can receive a vapor generated upon the heat transfer material undergoing the phase transition. In some embodiments, the third chamber comprises a capture material that captures the vapor. In some embodiments, the capture material comprises a hygroscopic material. In some embodiments, the capture material is a desiccant. In some embodiments, the activating comprises providing the capture material in the third chamber prior to (b).

In some embodiments, the third chamber is in fluid communication with a pump that draws the vapor. In some embodiments, the third chamber is in fluid communication with a fluid flow unit that subjects the vapor to flow from the third chamber to a vapor repository. In some embodiments, the fluid flow unit comprises a fan, a compressor and/or a pump. In some embodiments, the substrate comprises an additional heat transfer surface. In some embodiments, the method further comprises bringing a cooling fluid in contact with the additional heat transfer surface to subject the cooling fluid to cooling. In some embodiments, the cooling fluid comprises water or an alcohol. In some embodiments, the method further comprises using the cooling fluid to cool a reaction tube comprising the solution. In some embodiments, the activating in (a) comprises providing the heat transfer material in the first chamber. In some embodiments, the activating in (a) comprises bringing the second chamber in fluid communication with the first chamber.

An additional aspect of the disclosure provides a method for cooling a solution comprising a nucleic acid sample during a nucleic acid amplification reaction. The method comprises: (a) activating a heat exchange apparatus comprising: (1) a first chamber comprising a heat transfer material; (2) a second chamber comprising a substrate having a heat transfer surface, where the second chamber is fluidically isolated from the first chamber, and where the heat transfer surface is in thermal communication with the solution comprising the nucleic acid sample during the nucleic acid amplification reaction; and (3) a seal between the first chamber and the second chamber, which seal (i) isolates the second chamber from the first chamber when in a closed configuration, and (ii) brings the second chamber in fluid communication with the first chamber when in an open configuration. The method also comprises: (b) actuating the seal from the closed configuration to the open configuration to bring the first chamber in fluid communication with the second chamber. In the open configuration, the heat transfer material undergoes a phase transition that draws thermal energy from the substrate along the heat transfer surface to subject the solution to cooling.

In some embodiments, the method further comprises (c) subjecting the solution to cooling using the thermal energy drawn from the substrate along the heat transfer surface. In some embodiments, the heat transfer surface is in thermal communication with the solution indirectly through at least one heat transfer medium. In some embodiments, the heat transfer surface is in thermal communication with the solution directly. In some embodiments, the heat transfer material is a heat transfer liquid. In some embodiments, (b) comprises subjecting the heat transfer liquid to flow from the first chamber to the second chamber to come in contact with the heat transfer surface. In some embodiments, the apparatus further comprises a third chamber in fluid communication with the second chamber through at least one fluid flow path between the second chamber and the third chamber. The third chamber can receive a vapor generated upon the heat transfer material undergoing the phase transition.

In some embodiments, the third chamber comprises a capture material that captures the vapor. In some embodiments, the capture material comprises a hygroscopic material. In some embodiments, the capture material is a desiccant. In some embodiments, the activating comprises providing the capture material in the third chamber prior to (b). In some embodiments, the substrate comprises an additional heat transfer surface. In some embodiments, the method further comprises bringing a cooling fluid in contact with the additional heat transfer surface to subject the cooling fluid to cooling. In some embodiments, the method further comprises using the cooling fluid to cool a reaction tube comprising the solution. In some embodiments, the activating in (a) comprises providing the heat transfer material in the first chamber. In some embodiments, the activating in (a) comprises bringing the second chamber in fluid communication with the first chamber.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example apparatus for generating droplets;

FIG. 2 shows an enlarged view of part of an example apparatus for generating droplets;

FIG. 3 shows an example apparatus for fluidic cooling;

FIG. 4 shows an example system for cooling;

FIG. 5 shows an example system for fluidic cooling;

FIG. 6 shows an example nucleic acid amplification system with rotatable thermal zones;

FIG. 7 shows an example nucleic acid amplification system with rotatable thermal zones;

FIG. 8 (panels A and B) shows example nucleic acid amplification systems with rotatable thermal zones; and

FIG. 9 shows an example computer control system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an” and “the” include plural references unless the context clearly dictates otherwise. For example, the term “a molecule” includes a plurality of molecules, including mixtures thereof.

As used herein, the terms “amplifying” and “amplification” are used interchangeably and generally refer to generating one or more copies or “amplified product” of a nucleic acid. The term “DNA amplification” generally refers to generating one or more copies of a DNA molecule or “amplified DNA product”. The term “reverse transcription amplification” generally refers to the generation of deoxyribonucleic acid (DNA) from a ribonucleic acid (RNA) template via the action of a reverse transcriptase.

As used herein, the terms “denaturing” and “denaturation” are used interchangeably and generally refer to the full or partial unwinding of the helical structure of a double-stranded nucleic acid, and in some embodiments the unwinding of the secondary structure of a single stranded nucleic acid. Denaturation may include the inactivation of the cell wall(s) of a pathogen or the shell of a virus, and the inactivation of the protein(s) of inhibitors. Conditions at which denaturation may occur include a “denaturation temperature” that generally refers to a temperature at which denaturation is permitted to occur and a “denaturation duration” that generally refers to an amount of time allotted for denaturation to occur.

As used herein, the term “elongation” generally refers to the incorporation of nucleotides to a nucleic acid in a template directed fashion. Elongation may occur via the aid of an enzyme, such as, for example, a polymerase or reverse transcriptase. Conditions at which elongation may occur include an “elongation temperature” that generally refers to a temperature at which elongation is permitted to occur and an “elongation duration” that generally refers to an amount of time allotted for elongation to occur.

As used herein, the term “nucleic acid” generally refers to a polymeric form of nucleotides of any length, either deoxyribonucleotides (dNTPs) or ribonucleotides (rNTPs), or analogs thereof. Nucleic acids may have any three dimensional structure, and may perform any function, known or unknown. Non-limiting examples of nucleic acids include DNA, RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non nucleotide components. A nucleic acid may be further modified after polymerization, such as by conjugation or binding with a reporter agent.

As used herein, the term “primer extension reaction” generally refers to the denaturing of a double-stranded nucleic acid, binding of a primer to one or both strands of the denatured nucleic acid, followed by elongation of the primer(s).

As used herein, the term “reaction mixture” generally refers to a composition comprising reagents necessary to complete nucleic acid amplification (e.g., DNA amplification, RNA amplification), with non-limiting examples of such reagents that include primer sets having specificity for target RNA or target DNA, DNA produced from reverse transcription of RNA, a DNA polymerase, a reverse transcriptase (e.g., for reverse transcription of RNA), suitable buffers (including zwitterionic buffers), co-factors (e.g., divalent and monovalent cations), dNTPs, and other enzymes (e.g., uracil-DNA glycosylase (UNG)), etc). In some embodiments, reaction mixtures can also comprise one or more reporter agents.

As used herein, a “reporter agent” generally refers to a composition that yields a detectable signal, the presence or absence of which may be used to detect the presence of amplified product.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogues thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the term “subject,” generally refers to an entity or a medium that has testable or detectable genetic information. A subject may be a person or individual. A subject may be a vertebrate, such as, for example, a mammal. Non-limiting examples of mammals include murines, simians, humans, farm animals, sport animals, and pets. Other examples of subjects include, for example, food, plant, soil, and water.

As used herein, the term “fluid” generally refers to a liquid or a gas. A fluid cannot maintain a defined shape and will flow during an observable time frame to fill a container in which it is put. Thus, a fluid may have any suitable viscosity that permits flow. If two or more fluids are present, each fluid may be independently selected among essentially any fluids (liquids, gases, and the like).

As used herein, the term “aqueous fluid” generally refers to a fluid that is made with, of, or from water, or a fluid that contains water. For example, an aqueous fluid may be an aqueous solution with water as the solvent. An aqueous fluid of the present disclosure may comprise reagents necessary for conducting a desired chemical reaction, e.g., polymerase chain reaction (PCR). Non-limiting examples of aqueous fluid include, but are not limited to, water and other aqueous solutions comprising water, such as cell or biological medium, ethanol, salt solutions, etc.

As used herein, the term “continuous fluid” generally refers to a fluid that forms a continuous flow. A continuous fluid may be a fluid immiscible with an aqueous solution. For example, a continuous fluid may be a non-aqueous fluid made from, with, or using a liquid other than water. Non-limiting examples of continuous fluid include, but are not limited to, oils such as hydrocarbons, silicon oils, fluorine-containing oils (e.g., fluorocarbon oils), organic solvents etc.

As used herein, the term “channel” generally refers to a path that confines and/or directs the flow of a fluid. A channel of the present disclosure may be of any suitable length. The channel may be straight, substantially straight, or it may contain one or more curves, bends, etc. For example, the channel may have a serpentine or a spiral configuration. In some embodiments, the channel includes one or more branches, with some or all of which connected with one or more other channel(s).

As used herein, a “cross-sectional dimension” of a channel may be measured perpendicularly with respect to the general direction of fluid flow within the channel.

As used herein, the term “droplet” generally refers to an isolated portion of a first fluid (e.g., an aqueous fluid) that is surrounded by a second fluid (e.g., a continuous fluid). An emulsion may include a dispersion of droplets of a first fluid (e.g., liquid) in a second fluid. The first fluid may be immiscible with the second fluid. In some embodiments, the first fluid and the second fluid are substantially immiscible. A droplet of the present disclosure may be spherical or assume other shapes, such as, for example, shapes with elliptical cross-sections. The diameter of a droplet, in a non-spherical droplet, is the diameter of a perfect mathematical sphere having the same volume as the non-spherical droplet. A droplet may include a skin. The skin may form upon heating the droplet. The skin may have a higher viscosity than an interior of the droplet. In some embodiments, the skin may prevent the droplet from fusing with other droplets.

As used herein, the term “sample” generally refers to any sample containing or suspected of containing a nucleic acid molecule. For example, a subject sample may be a biological sample containing one or more nucleic acid molecules. The biological sample may be obtained (e.g., extracted or isolated) from a bodily sample of a subject that may be selected from blood (e.g., whole blood), plasma, serum, urine, saliva, mucosal excretions, sputum, stool and tears. The bodily sample may be a fluid or tissue sample (e.g., skin sample) of the subject. In some examples, the sample is obtained from a cell-free bodily fluid of the subject, such as whole blood. In such instance, the sample can include cell-free DNA and/or cell-free RNA. In some other examples, the sample is an environmental sample (e.g., soil, waste, ambient air and etc.), industrial sample (e.g., samples from any industrial processes), and food samples (e.g., dairy products, vegetable products, and meat products).

In some embodiments, a sample is obtained directly from a subject without further processing. In some embodiments, a sample is processed prior to a biological or chemical reaction (e.g., nucleic acid amplification). For example, a lysis agent may be added to a sample holder prior to adding a biological sample and reagents necessary for nucleic acid amplification. Examples of the lysis agent include Tris-HCl, EDTA, detergents (e.g., Triton X-100, SDS), lysozyme, glucolase, proteinase E, viral endolysins, exolysins zymolose, Iyticase, proteinase K, endolysins and exolysins from bacteriophages, endolysins from bacteriophage PM2, endolysins from the B. subtilis bacteriophage PBSX, endolysins from Lactobacillus prophages Lj928, Lj965, bacteriophage 15 Phiadh, endolysin from the Streptococcus pneumoniae bacteriophage Cp-I, bifunctional peptidoglycan lysin of Streptococcus agalactiae bacteriophage B30, endolysins and exolysins from prophage bacteria, endolysins from Listeria bacteriophages, holin-endolysin, cell 20 lysis genes, holWMY Staphylococcus wameri M phage varphiWMY, Iy5WMY of the Staphylococcus wameri M phage varphiWMY, Tween 20, PEG, KOH, NaCl, and combinations thereof. In some embodiments, a lysis agent is sodium hydroxide (NaOH). In some embodiments, the biological sample is not treated with a detergent.

In some embodiments, the sample is purified (e.g., by filtration, centrifugation, column purification and/or magnetic purification, for example, by using magnetic beads (e.g., super paramagnetic beads)) to obtain purified nucleic acids.

As used herein, the term “about” or “nearly” generally refers to a reasonable variation, e.g. within +/−10%, 9%, 8%, 7%, 6%, 5%, 4%, 3%, 2%, or 1% of a designated amount.

As used herein, the term “overshooting” generally refers to a point or region that is above or below a target or designated point or region. In some examples, in heating, an overshooting thermal zone may be at a temperature that is above a target temperature, and in cooling, an overshooting thermal zone may be at a temperature that is below a target temperature. For example, in heating a solution to 100° C., an overshooting thermal zone at a temperature of about 140° C. may be used. In another example, in cooling a solution to 25° C., an overshooting thermal zone at a temperature of about 0° C. may be used. An overshooting thermal zone may provide a greater temperature drop or temperature change, which may in turn provide a greater rate of heat transfer to provide heating or cooling, as necessary or required.

As used herein, the term “thermal communication” generally refers to a state in which two or more materials are capable of exchange energy, such as thermal energy, with one another. Such exchange of energy may be by way of transfer of energy from one material to another material. Such transfer of energy may be radiative, conductive, or convective heat transfer. The energy may be thermal energy. In some examples, two or more materials that are in thermal communication with one another are in thermal contact with one another, such as, for example, direct physical contact or contact through one or more intermediary materials.

### Droplet Generation

In an aspect, the present disclosure provides an apparatus for generating at least one droplet comprising a biological sample for use in a chemical or biological reaction. The apparatus may comprise a first chamber comprising a first fluid volume and at least one first fluid flow port that is in fluid communication with the first fluid volume. The first fluid volume may retain an aqueous solution comprising the biological sample for use in the chemical or biological reaction. The apparatus may comprise a second chamber comprising a second fluid volume and at least one second fluid flow port that is in fluid communication with the second fluid volume, the second chamber may at least partially circumscribe the first chamber. In some embodiments, the second chamber fully circumscribes the first chamber. The second fluid volume may retain a continuous fluid that is immiscible with the aqueous solution, and the second chamber may be rotatable with respect to the first chamber, or vice versa. In some embodiments, the second chamber is rotatable with respect to the first chamber. In some embodiments, the first chamber is rotatable with respect to the second chamber. The first chamber and/or the second chamber may be cylindrical.

During use, rotation of the first chamber or the second chamber may bring the first fluid flow port in alignment with the second fluid flow port to subject the aqueous solution comprising the biological sample to flow from the first fluid volume to the second fluid volume to generate the at least one droplet upon contact with the continuous fluid, and the at least one droplet may comprise the biological sample or a portion thereof. The first fluid flow port may be in selective alignment with the second fluid flow port upon rotation of the first chamber or the second chamber. The at least one droplet may comprise a plurality of droplets, and each of the plurality of droplets may comprise the biological sample or a portion thereof.

In another aspect, the present disclosure provides a method for generating at least one droplet comprising a biological sample for use in a chemical or biological reaction. The method may comprise: (a) activating an apparatus comprising (1) a first chamber comprising a first fluid volume and at least one first fluid flow port that is in fluid communication with the first fluid volume, and (2) a second chamber comprising a second fluid volume and at least one second fluid flow port that is in fluid communication with the second fluid volume, the first fluid volume may comprise an aqueous solution comprising the biological sample for use in the chemical or biological reaction, and the second chamber may at least partially circumscribe the first chamber. In some embodiments, the second chamber fully circumscribes the first chamber. The second fluid volume may retain a continuous fluid that is immiscible with the aqueous solution, and the second chamber may be rotatable with respect to the first chamber, or vice versa. The method may further comprise (b) rotating the first chamber or the second chamber to bring the first fluid flow port in alignment with the second fluid flow port to subject the aqueous solution comprising the biological sample to flow from the first fluid volume to the second fluid volume to generate the at least one droplet upon contact with the continuous fluid. The rotating in (b) may comprise rotating the second chamber with respect to the first chamber, or rotating the first chamber with respect to the second chamber. The at least one droplet may comprise the biological sample or a portion thereof. The activating may comprise depositing the aqueous solution comprising the biological sample in the first fluid volume.

In various aspects of the present disclosure, the at least one first fluid flow port and/or the at least one second fluid flow port may be dimensioned such that the at least one droplet has a predetermined characteristic size and/or shape. The at least one first fluid flow port may comprise a plurality of first fluid flow ports. The plurality of first fluid flow ports may be brought in alignment with the at least one second fluid flow port upon rotation of the first chamber or the second chamber. The at least one second fluid flow port may comprise a plurality of second fluid flow ports. The plurality of second fluid flow ports may be brought in alignment with the at least one first fluid flow port upon rotation of the first chamber or the second chamber. In some embodiments, the second chamber may be fixed and the first chamber may rotate to bring the at least one first fluid flow port in alignment with the at least one second fluid flow port. As an alternative, the first chamber may be fixed and the second chamber may rotate to bring the at least one second fluid flow port in alignment with the at least one first fluid flow port. In some embodiments, both the first chamber and the second chamber may rotate, either in the same direction or in opposite directions, to bring the at least one first fluid flow port in alignment with the at least one second fluid flow port.

The biological sample may be any suitable sample of a subject. For example, the biological sample may be solid matter (e.g., biological tissue) or may be a fluid (e.g., a biological fluid). In general, a biological fluid can include any fluid associated with living organisms. Non-limiting examples of a biological sample include blood (or components of blood, e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, microbiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues.

The biological sample may be obtained from a subject in a variety of ways. Non-limiting examples of approaches to obtain a biological sample from a subject include accessing the circulatory system (e.g., intravenously or intra-arterially via a syringe or other needle), collecting a secreted biological sample (e.g., feces, urine, sputum, saliva, etc.), surgically (e.g., biopsy), swabbing (e.g., buccal swab, oropharyngeal swab), pipetting, and breathing. Moreover, a biological sample may be obtained from any anatomical part of a subject where a desired biological sample is located.

In some embodiments, the biological sample is from a genome of the subject. In some embodiments, the biological sample is a cell-free nucleic acid sample. For example, the biological sample may be cell-free deoxyribonucleic acid (DNA) or cell-free ribonucleic acid (RNA).

The biological sample may be obtained directly from the subject. A biological sample obtained directly from a subject may be a biological sample that has not been further processed after being obtained from the subject, with the exception of any approach used to collect the biological sample from the subject for further processing. For example, blood is obtained directly from a subject by accessing the subject's circulatory system, removing the blood from the subject (e.g., via a needle), and entering the removed blood into a receptacle. The receptacle may comprise reagents (e.g., anti-coagulants) such that the blood sample is useful for further analysis. In another example, a swab may be used to access epithelial cells on an oropharyngeal surface of the subject. After obtaining the biological sample from the subject, the swab containing the biological sample may be contacted with a fluid (e.g., a buffer) to collect the biological fluid from the swab. In some embodiments, the biological sample is obtained directly from the subject and provided in the first channel without sample purification and/or ribonucleic acid (RNA) extraction. For example, the RNA or DNA in a biological sample may not be extracted from the biological sample when providing the sample in the first chamber and/or the aqueous solution. Moreover, in some embodiments, a target nucleic acid (e.g., a target RNA or target DNA) present in a biological sample is not concentrated prior to providing the biological sample to the aqueous solution and/or the first chamber.

The at least one droplet may comprise a plurality of droplets, and each of the plurality of droplets may comprise the biological sample or a portion thereof.

The at least one droplet may have a size that is at least partially dependent on a rate of rotation of the first chamber or the second chamber. The rate of droplet formation may be at least partially dependent on a rate of rotation of the first chamber or the second chamber. For example, when the rate of rotation of the first chamber or the second chamber is high, the rate of droplet formation may be high, and when the rate of rotation of the first chamber or the second chamber is low, the rate of droplet formation may be low. In another example, when the rate of rotation of the first chamber or the second chamber is high, the droplet may have a smaller size, and when the rate of rotation of the first chamber or the second chamber is low, the droplet may have a bigger size.

A droplet of the present disclosure may be an isolated portion of a first fluid (e.g., an aqueous solution) that is completely surrounded by a second fluid (e.g., a continuous fluid). A droplet may be of any suitable shape and it may not necessarily be spherical. The diameter of a droplet, in a non-spherical droplet, is the diameter of a perfect mathematical sphere having the same volume as the non-spherical droplet.

droplet of the present disclosure may be formed when a portion of a first fluid (e.g., an aqueous fluid) is substantially surrounded by a second fluid (e.g., a continuous fluid). As used herein, a portion of a first fluid is “surrounded” by a second fluid when a closed loop may be drawn around the first fluid through only the second fluid. A portion of a first fluid is “completely surrounded” by a second fluid if closed loops going through only the second fluid may be drawn around the first fluid regardless of direction. A portion of a first fluid is “substantially surrounded” by a second fluid if the loops going through only the second fluid may be drawn around the droplet depending on the direction.

An average size of the droplet may depend on the properties (e.g. flow rate, viscosity) of one or more of the fluids, the size, configuration, or geometry of the chambers, and/or the size, configuration, or geometry of the fluid flow ports.

The first chamber may be of any shape and/or dimension suitable for holding an aqueous solution comprising a biological sample. For example, the first chamber may be cone shaped, cubic, cylindrical, or of any other suitable shapes. The first chamber may be dimensioned to hold a first fluid volume of at least or about 0.1 microliters (μl), 0.5 μl, 1 μl, 1.5 μl, 2 μl, 2.5 μl, 3 μl, 3.5 μl, 4 μl, 4.5 μl, 5 μl, 5.5 μl, 6 μl, 6.5 μl, 7 μl, 7.5 μl, 8 μl, 8.5 μl, 9 μl, 9.5 μl, 10 μl, 11 μl, 12 μl, 13 μl, 14 μl, 15 μl, 16 μl, 17 μl, 18 μl, 19 μl, 20 μl, 21 μl, 22 μl, 23 μl, 24 μl, 25 μl, 26 μl, 27 μl, 28 μl, 29 μl, 30 μl, 35 μl, 40 μl, 45 μl, 50 μl, 55 μl, 60 μl, 65 μl, 70 μl, 75 μl, 80 μl, 85 μl, 90 μl, 95 μl, 100 μl, 110 μl, 120 μl, 130 μl, 140 μl, 150 μl, 160 μl, 170 μl, 180 μl, 190 μl, 200 μl, 300 μl, 400 μl, 500 μl, 600 μl, 700 μl, 800 μl, 900 μl, 1000 μl, 2 ml, 3 ml, 4 ml, 5 ml, 6 ml, 7 ml, 8 ml, 9 ml, or more.

The first chamber may comprise one or more first fluid flow ports. At least one of the one or more first fluid ports is in fluid communication with the first fluid volume. For example, an aqueous solution comprising a biological sample may flow in and/or out of the first chamber through the one or more first fluid flow ports. The one or more first fluid flow ports may be of the same or of different shapes, and they may be of the same or different dimensions. For example, each of the one or more first fluid flow ports may independently have a diameter of no more than about 1 millimeter (mm), no more than about 800 micrometers (μm), no more than about 600 μm, no more than about 500 μm, no more than about 400 μm, no more than about 300 μm, no more than about 250 μm, no more than about 200 μm, no more than about 100 μm, no more than about 75 μm, no more than about 50 μm, no more than about 25 μm, no more than about 10 μm, no more than about 5 μm, no more than about 4 μm, no more than about 3 μm, no more than about 2 μm, no more than about 1 μm, or less. In some embodiments, each of the one or more first fluid flow ports may independently have a diameter of at least about 1 μm, at least about 2 μm, at least about 3 μm, at least about 4 μm, at least about 5 μm, at least about 10 μm, at least about 15 μm, at least about 20 μm, at least about 25 μm, at least about 50 μm, at least about 75 μm, at least about 100 μm, at least about 200 μm, at least about 250 μm, at least about 300 μm, at least about 400 μm, at least about 500 μm, at least about 600 μm, at least about 800 μm, or more. In some embodiments, each of the one or more first fluid flow ports may have a diameter that is greater than a cross-section of the at least one droplet.

The second chamber may be of any shape and/or dimension suitable for holding a continuous fluid. For example, the second chamber may be cone shaped, cubic, cylindrical, or of any other suitable shapes. The second chamber may at least partially circumscribe the first chamber. In some embodiments, the second chamber completely circumscribes the first chamber. For example, the first chamber may be placed inside the second chamber. The second chamber may be dimensioned to hold a second fluid volume of at least or about 0.1 μl, 0.5 μl, 1 μl, 1.5 μl, 2 μl, 2.5 μl, 3 μl, 3.5 μl, 4 μl, 4.5 μl, 5 μl, 5.5 μl, 6 μl, 6.5 μl, 7 μl, 7.5 μl, 8 μl, 8.5 μl, 9 μl, 9.5 μl, 10 μl, 11 μl, 12 μl, 13 μl, 14 μl, 15 μl, 16 μl, 17 μl, 18 μl, 19 μl, 20 μl, 21 μl, 22 μl, 23 μl, 24 μl, 25 μl, 26 μl, 27 μl, 28 μl, 29 μl, 30 μl, 35 μl, 40 μl, 45 μl, 50 μl, 55 μl, 60 μl, 65 μl, 70 μl, 75 μl, 80 μl, 85 μl, 90 μl, 95 μl, 100 μl, 110 μl, 120 μl, 130 μl, 140 μl, 150 μl, 160 μl, 170 μl, 180 μl, 190 μl, 200 μl, 300 μl, 400 μl, 500 μl, 600 μl, 700 μl, 800 μl, 900 μl, 1000 μl, 2 ml, 3 ml, 4 ml, 5 ml, 6 ml, 7 ml, 8 ml, 9 ml, 10 mL, 11 mL, 12 mL, 13 mL, 14 mL, 15 mL, 16 mL, 17 mL, 18 mL, 19 mL, 20 mL, 25 mL, 30 mL or more.

The second chamber may comprise one or more second fluid flow ports. At least one of the one or more second fluid ports is in fluid communication with the second fluid volume. For example, a continuous fluid immiscible with the aqueous solution may flow in and/or out of the second chamber through the one or more second fluid flow ports. The one or more second fluid flow ports may be of the same or of different shapes, and they may be of the same or different dimensions. For example, each of the one or more second fluid flow ports may independently have a diameter of no more than about 5 cm, no more than about 3 cm, no more than about 1 cm, no more than about 900 mm, no more than about 800 mm, no more than about 700 mm, no more than about 600 mm, no more than about 500 mm, no more than about 400 mm, no more than about 300 mm, no more than about 200 mm, no more than about 100 mm, no more than about 75 mm, no more than about 50 mm, no more than about 25 mm, no more than about 10 mm, no more than about 5 mm, no more than about 1 mm, no more than about 900 μm, no more than about 800 μm, no more than about 700 μm, no more than about 600 μm, no more than about 500 μm, no more than about 400 μm, no more than about 300 μm, no more than about 250 μm, no more than about 200 μm, no more than about 100 μm, no more than about 75 μm, no more than about 50 μm, no more than about 25 μm, no more than about 10 μm, no more than about 5 μm, no more than about 1 μm or less. In some embodiments, each of the one or more second fluid flow ports may independently have a diameter of at least about at least about 2 μm, at least about 3 μm, at least about 4 μm, 5 μm, at least about 10 μm, at least about 15 μm, at least about 20 μm, at least about 25 μm, at least about 50 μm, at least about 75 μm, at least about 100 μm, at least about 200 μm, at least about 250 μm, at least about 300 μm, at least about 400 μm, at least about 500 μm, at least about 600 μm, at least about 700 μm, at least about 800 μm, at least about 900 μm, at least about 1 mm, at least about 5 mm, at least about 10 mm, at least about 25 mm, at least about 50 mm, at least about 75 mm, at least about 100 mm, at least about 200 mm, at least about 300 mm, at least about 400 mm, at least about 500 mm, at least about 600 mm, at least about 700 mm, at least about 800 mm, at least about 900 mm, at least about 1 cm, at least about 3 cm, at least about 5 cm, or more. In some embodiments, each of the one or more second fluid flow ports may have a diameter that is greater than a cross-section of the at least one droplet.

A first fluid flow port is in alignment with a second fluid flow port when at least a part of the first fluid flow port is positioned in line with at least a part of the second fluid flow port, thereby enabling fluid passing through the first fluid flow port to enter the second fluid flow port, and/or enabling fluid passing through the second fluid flow port to enter the first fluid flow port. In some embodiments, a first fluid flow port may be in selective alignment with a second fluid flow port. For example, a first fluid flow port is configured to align with some of the second fluid flow ports but not the others. In some embodiments, a first fluid flow port may be aligned with a second fluid flow port only under one or more specific conditions (e.g., at a predetermined time point, under a specific pressure, when moving at a specific speed, at a periodic time-point, when generating droplets of a given size, etc.).

The first fluid volume may be in fluid communication with a source of positive pressure that subjects the aqueous solution to flow from the first fluid volume to the second fluid volume when the first fluid flow port is aligned with the second fluid flow port, to generate the one or more droplets upon contact with the continuous fluid. The source of positive pressure may subject the first chamber or the second chamber to rotation. The source of positive pressure may be a compressor. Other sources of a positive pressure may include a pump, gravity, capillary action, surface tension, electroosmosis, centrifugal forces, etc. In some embodiments, the source of positive pressure is a plunger. The plunger may be actuated by a user, and/or by a mechanical unit to generate positive pressure. For example, a user may directly or indirectly apply a force to a plunger pump to push an aqueous solution comprising a biological sample to flow through the plunger pump into the first chamber, the user may apply a further force to push the aqueous solution to flow from the first chamber into the second chamber comprising a continuous solution (e.g., an oil) through a first fluid flow port properly aligned with a second fluid flow port, thereby generating one or more droplets.

The second fluid volume may be in fluid communication with a source of negative pressure that subjects the aqueous solution to flow from the first fluid volume to the second fluid volume when the first fluid flow port is aligned with the second fluid flow port, to generate the one or more droplets upon contact with the continuous fluid. For example, a vacuum (e.g., from a vacuum pump or other suitable vacuum source) may be applied as a source of negative pressure to pull the aqueous solution to flow from the first chamber into the second chamber through a first fluid flow port properly aligned with a second fluid flow port, thereby generating one or more droplets.

The continuous fluid may comprise an oil. The oil may be a fluorine-containing oil. For example, the oil may be a fluorocarbon oil. For example, the continuous fluid may comprise hydrophobic liquids. Non-limiting examples of the hydrophobic liquids include oils, such as hydrocarbons, silicon oils, fluorocarbon oils, organic solvents etc. In some embodiments, the oil is a fluorinated oil, such as HFE 7500, FC-40, FC-43, FC-70, or a combination thereof.

The continuous fluid may comprise a surfactant. The surfactant may comprise a hydrophobic tail and a hydrophilic head group, a polymer-based tail and a hydrophilic head group, a polymer-based tail and a polymer-based head group, a fluorinated tail and a hydrophilic head group, or a fluorinated polymer-based tail and a hydrophilic polymer-based head group. In some embodiments, the surfactant is of a di-block copolymer or tri-block copolymer type. In some embodiments, the surfactant is a fluorinated surfactant. For example, the surfactant may be a block copolymer, such as a tri-block copolymer consisting of two perfluoropolyether blocks and one poly(ethylene)glycol block. In some embodiments, the surfactant is selected from the group consisting of PFPE-PEG-PFPE (perfluoropolyether-polyethylene glycol-perfluoropolyether), tri-block copolymer EA-surfactant (RainDance Technologies) and DMP (dimorpholino phosphate)-surfactant (Baret, Kleinschmidt, et al., 2009). The surfactant may be present in the continuous fluid with a concentration of 0.0001% to 5% (w/w), e.g., 0.001% to 4% (w/w), 0.01% to 3% (w/w), 0.1% to 2% (w/w), 0.1% to 1% (w/w). In some embodiments, the surfactant is present in the non-aqueous fluid with a concentration of at least about 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1.0% (w/w), 1.5% (w/w), 2.0% (w/w), 2.5% (w/w), 3.0% (w/w), 3.5% (w/w), 4.0% (w/w), 4.5% (w/w), 5.0% (w/w) or more.

The aqueous solution may comprise reagents necessary for the chemical or biological reaction. The chemical or biological reaction may be nucleic acid amplification, and the reagents may include one or more primers and polymerizing enzymes. For example, the nucleic acid amplification may be polymerase chain reaction (PCR).

A variety of nucleic acid amplification reactions may be used to amplify a target nucleic acid in the biological sample and generate an amplified product. Moreover, amplification of a nucleic acid may be linear, exponential, or a combination thereof. Non-limiting examples of nucleic acid amplification methods include reverse transcription, primer extension, polymerase chain reaction, ligase chain reaction, helicase-dependent amplification (e.g., amplification that is preceded by contacting the nucleic acid with a helicase), asymmetric amplification, rolling circle amplification, and multiple displacement amplification (MDA). In some embodiments, the amplified product may be DNA. In cases where a target RNA is amplified, DNA may be obtained by reverse transcription of the RNA and subsequent amplification of the DNA may be used to generate an amplified DNA product. The amplified DNA product may be indicative of the presence of the target RNA in the biological sample. In cases where DNA is amplified, one or more DNA amplification approaches may be employed. Non-limiting examples of DNA amplification approaches include polymerase chain reaction (PCR), variants of PCR (e.g., real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, touchdown PCR), and ligase chain reaction (LCR). In some embodiments, DNA amplification is linear. In some embodiments, DNA amplification is exponential. In some embodiments, DNA amplification is achieved with nested PCR, which can improve sensitivity of detecting amplified DNA products.

In any of the various aspects, nucleic acid amplification reactions described herein may be conducted in parallel. In general, parallel amplification reactions are amplification reactions that occur in the same droplet and at the same time. Parallel nucleic acid amplification reactions may be conducted, for example, by including reagents necessary for each nucleic acid amplification reaction in a droplet to obtain a reaction mixture and subjecting the reaction mixture to conditions necessary for each nucleic amplification reaction. For example, reverse transcription amplification and DNA amplification may be conducted in parallel, by providing reagents necessary for both amplification methods in a droplet to obtain a reaction mixture and subjecting the reaction mixture to conditions suitable for conducting both amplification reactions. DNA generated from reverse transcription of the RNA may be amplified in parallel to generate an amplified DNA product. Any suitable number of nucleic acid amplification reactions may be conducted in parallel. In some embodiments, at least 1, 2, 3, 4, 5, 6, 7, 8, 9, 10, 11, 12, 13, 14, 15, 16, 17, 18, 19, 20, 30, 40, 50, 100, 200, 300, 400, 500, 1000, 10,000, or more nucleic acid amplification reactions are conducted in parallel.

The reagents necessary for nucleic acid amplification may include a polymerizing enzyme and primers having sequence complementary with a target nucleic acid sequence.

In any of the various aspects, primers sets directed to a target nucleic acid may be utilized to conduct nucleic acid amplification reaction. Primer sets generally comprise one or more primers. For example, a primer set may comprise about 1, 2, 3, 4, 5, 6, 7, 8, 9, 10, 20, 30, 40, 50, or more primers. In some embodiments, a primer set comprises primers directed to different amplified products or different nucleic acid amplification reactions. For example, a primer set may comprise a first primer necessary to generate a first strand of nucleic acid product that is complementary to at least a portion of the target nucleic acid and a second primer complementary to the nucleic acid strand product necessary to generate a second strand of nucleic acid product that is complementary to at least a portion of the first strand of nucleic acid product.

For example, a primer set may be directed to a target RNA. The primer set may comprise a first primer that may be used to generate a first strand of nucleic acid product that is complementary to at least a portion the target RNA. In the case of a reverse transcription reaction, the first strand of nucleic acid product may be DNA. The primer set may also comprise a second primer that may be used to generate a second strand of nucleic acid product that is complementary to at least a portion of the first strand of nucleic acid product. In the case of a reverse transcription reaction conducted in parallel with DNA amplification, the second strand of nucleic acid product may be a strand of nucleic acid (e.g., DNA) product that is complementary to a strand of DNA generated from an RNA template.

Where desired, any suitable number of primer sets may be used. For example, about 1, 2, 3, 4, 5, 6, 7, 8, 9, 10, or more primer sets may be used. Where multiple primer sets are used, one or more primer sets may each correspond to a particular nucleic acid amplification reaction or amplified product.

In some embodiments, a DNA polymerase is used. Any suitable DNA polymerase may be used, including commercially available DNA polymerases. A DNA polymerase generally refers to an enzyme that is capable of incorporating nucleotides to a strand of DNA in a template bound fashion. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at a temperature from about 92° C. to 95° C. (e.g., about 94° C. to 95° C.) for a time period from about 2 minutes to 10 minutes may be required, which may change the thermal profile based on different polymerases.

In some embodiments, a reverse transcriptase is used. Any suitable reverse transcriptase may be used. A reverse transcriptase generally refers to an enzyme that is capable of incorporating nucleotides to a strand of DNA, when bound to an RNA template. Non-limiting examples of reverse transcriptases include HIV-1 reverse transcriptase, M-MLV reverse transcriptase, AMV reverse transcriptase, telomerase reverse transcriptase, and variants, modified products and derivatives thereof.

The target nucleic acid sequence may be associated with a disease. The disease may be associated with a virus such as for example an RNA virus or a DNA virus. In some embodiments, the virus may be selected from the group consisting of human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, and Varicella virus. In some embodiments, the influenza virus is selected from the group consisting of H1N1 virus, H3N2 virus, H7N9 virus and H5N1 virus. In some embodiments, the adenovirus is adenovirus type 55 (ADV55) or adenovirus type 7 (ADV7). In some embodiments, the hepatitis C virus is armored RNA-hepatitis C virus (RNA-HCV). In some embodiments, the disease is associated with a pathogenic bacterium (e.g., Mycobacterium tuberculosis) or a pathogenic protozoan (e.g., Plasmodium).

In some embodiments, the disease is cancer. Non-limiting examples of the cancers include colorectal cancer, bladder cancer, ovarian cancer, testicular cancer, breast cancer, skin cancer, lung cancer, pancreatic cancer, stomach cancer, esophageal cancer, brain cancer, leukemia, liver cancer, endometrial cancer, prostate cancer, and head and neck cancer.

The droplets may include detectable moieties that permit detection of any signals generated from the biological and/or chemical reactions (e.g., nucleic acid amplification reactions). For example, the detectable moieties may yield a detectable signal whose presence or absence is indicative of a presence of an amplified product. The intensity of the detectable signal may be proportional to the amount of amplified product. In some embodiments, where amplified product is generated of a different type of nucleic acid than the target nucleic acid initially amplified, the intensity of the detectable signal may be proportional to the amount of target nucleic acid initially amplified. For example, in the case of amplifying a target RNA via parallel reverse transcription and amplification of the DNA obtained from reverse transcription, reagents necessary for both reactions may also comprise a detectable moiety that yield a detectable signal indicative of the presence of the amplified DNA product and/or the target RNA amplified. The intensity of the detectable signal may be proportional to the amount of the amplified DNA product and/or the original target RNA amplified. The use of a detectable moiety also enables real-time amplification methods, including real-time PCR for DNA amplification.

Detectable moieties may be linked with nucleic acids, including amplified products, by covalent or non-covalent interactions. Non-limiting examples of non-covalent interactions include ionic interactions, Van der Waals forces, hydrophobic interactions, hydrogen bonding, and combinations thereof. In some embodiments, detectable moieties bind to initial reactants and changes in detectable moiety levels are used to detect amplified product. In some embodiments, detectable moieties are only detectable (or non-detectable) as nucleic acid amplification progresses. In some embodiments, an optically-active dye (e.g., a fluorescent dye) is used as a detectable moiety. Non-limiting examples of dyes include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

In some embodiments, a detectable moiety is a sequence-specific oligonucleotide probe that is optically active when hybridized with an amplified product. Due to sequence-specific binding of the probe to the amplified product, use of oligonucleotide probes can increase specificity and sensitivity of detection. A probe may be linked to any of the optically-active detectable moieties (e.g., dyes) described herein and may also include a quencher capable of blocking the optical activity of an associated dye. Non-limiting examples of probes that may be useful as detectable moieties include TaqMan probes, TaqMan Tamara probes, TaqMan MGB probes, or Lion probes.

In some embodiments and where a detectable moiety is an RNA oligonucleotide probe that includes an optically-active dye (e.g., fluorescent dye) and a quencher positioned adjacently on the probe. The close proximity of the dye with the quencher can block the optical activity of the dye. The probe may bind to a target sequence to be amplified. Upon the breakdown of the probe with the exonuclease activity of a DNA polymerase during amplification, the quencher and dye are separated, and the free dye regains its optical activity that can subsequently be detected.

In some embodiments, a detectable moiety is a molecular beacon. A molecular beacon includes, for example, a quencher linked at one end of an oligonucleotide in a hairpin conformation. At the other end of the oligonucleotide is an optically active dye, such as, for example, a fluorescent dye. In the hairpin configuration, the optically-active dye and quencher are brought in close enough proximity such that the quencher is capable of blocking the optical activity of the dye. Upon hybridizing with amplified product, however, the oligonucleotide assumes a linear conformation and hybridizes with a target sequence on the amplified product. Linearization of the oligonucleotide results in separation of the optically-active dye and quencher, such that the optical activity is restored and may be detected. The sequence specificity of the molecular beacon for a target sequence on the amplified product can improve specificity and sensitivity of detection.

In some embodiments, a detectable moiety is a radioactive species. Non-limiting examples of radioactive species include ¹⁴C, ¹²³I, ¹²⁴I, ¹²⁵I, ¹³¹I, Tc⁹⁹m, ³⁵S, and ³H.

In some embodiments, a detectable moiety is an enzyme that is capable of generating a detectable signal. Detectable signal may be produced by activity of the enzyme with its substrate or a particular substrate in the case the enzyme has multiple substrates. Non-limiting examples of enzymes that may be used as detectable moieties include alkaline phosphatase, horseradish peroxidase, I²-galactosidase, alkaline phosphatase, β-galactosidase, acetylcholinesterase, and luciferase.

The second chamber may comprise an inner partition and an outer partition circumscribing the inner partition. The inner partition and the outer partition may at least partially define the second fluid volume, and the inner partition may be adjacent to the first chamber. In some embodiments, the inner partition is in contact with the first chamber.

The apparatus may further comprise a fluid flow path between the first chamber and the inner partition. The aqueous solution may not be subjected to flow from the first fluid volume to the second fluid volume in the absence of the first fluid flow port being in alignment with the second fluid flow port. For example, when the first fluid flow port is misaligned with the second fluid flow port, the aqueous solution may not be able to flow from the first fluid volume to the second fluid volume.

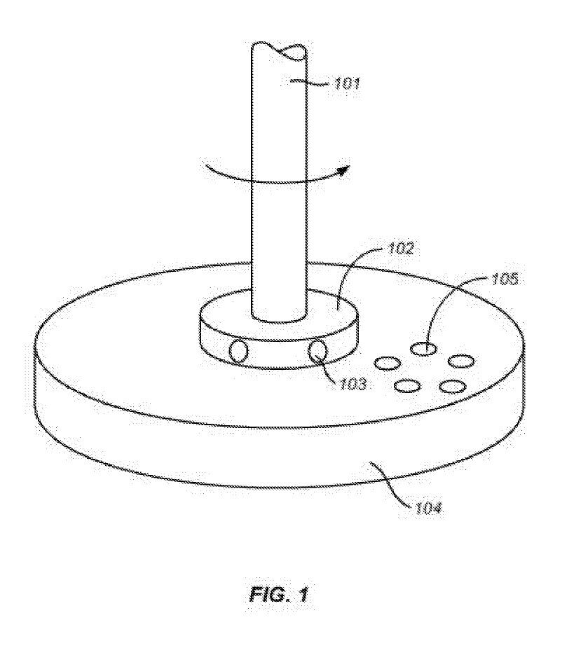

FIG. 1 provides an example of an apparatus for droplet generation. A plunger 101 is in fluid communication with a first chamber 102, an aqueous solution comprising a biological sample may be pumped through the plunger 101 to enter the first chamber 102. A second chamber 104 comprising a continuous solution substantially immiscible with the aqueous solution circumscribes the first chamber 102. The first chamber 102 and the second chamber 104 each comprises several fluid flow ports, which are in fluid communication with the first chamber 102 or the second chamber 104, respectively. Actuation (e.g., vertical motion or rotation) of the plunger 101 may subject the first chamber 102 to rotation, and rotation of the first chamber 102 may bring the fluid flow ports of the first chamber 102 in alignment with the fluid flow ports of the second chamber 104, thereby forming aligned fluid flow ports 103. The aqueous solution comprising a biological sample may then flow from the first chamber 102 into the second chamber 104 through the aligned fluid flow ports 103 to generate droplets 105 up on contact with the continuous fluid in the second chamber 104.

The first chamber 102 may rotate in relation to the second chamber 104. As an alternative, second chamber 104 may rotate in relation to the first chamber 102. As another alternative, both the first chamber 102 and second chamber 104 may rotate. Rotation of the first chamber 102 and/or the second chamber 104 brings fluid flow ports in alignment, such that the aqueous solution may flow form the first chamber 102 to the second chamber to form one or more droplets.

The apparatus may include one or more fluid flow ports for permitting the aqueous solution to flow from the first chamber 102 into the second chamber 104. For example, the apparatus can include at least 1, 2, 3, 4, 5, 6, 7, 8, 9, 10, 20, 30, 40, 50, 100, 200, 300, 400, or 500 fluid flow ports. The fluid flow ports may have various configurations. For example, the fluid flow ports may be distributed around a circumference of the first chamber 104. A subset of the fluid flow ports may be on top of one another along a longitudinal axis of the plunger 101.

The first chamber 102 and/or the second chamber 104 can be rotated with the aid of one or more actuators, such as a motor. As an alternative, the first chamber 102 and/or the second chamber 104 can be rotated upon the plunger 101 being moved vertically up and/or down, which can provide, for example, positive pressure to subject the first chamber 102 and/or the second chamber 104 to rotation. For example, vertical movement of the plunger 101 can decrease a volume of the first chamber 102, which can increase pressure in the first chamber and subject the first chamber 102 to rotation.

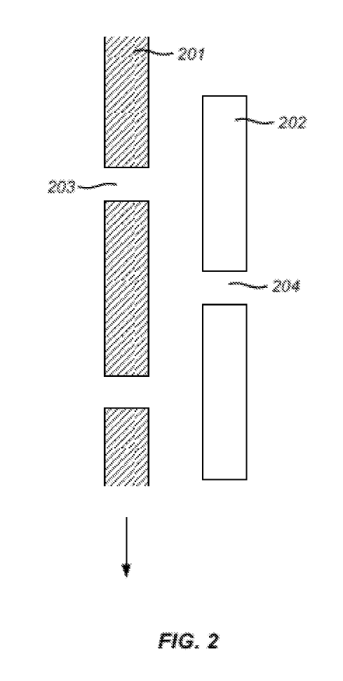

FIG. 2 illustrates alignment of fluid flow ports in an apparatus for droplet generation. A container wall 201 of a first chamber may comprise multiple first fluid flow ports 203, and a container wall 202 of a second chamber may comprise multiple second fluid flow ports 204. Rotation of the first chamber in a direction indicated by the arrow may bring the first fluid flow ports 203 in alignment with the second fluid flow ports 204, thereby allowing a first fluid within the first chamber to flow into the second chamber through the aligned fluid flow ports.

### Rotatable Thermal Zones for Nucleic Acid Amplification

In an aspect, the present disclosure provides a system for conducting a chemical or biological reaction on a biological sample. The system may comprise a sample holder that receives a solution comprising the biological sample, the sample holder may retain the solution during the chemical or biological reaction. The system may further comprise a plurality of thermal zones comprising at least a first thermal zone and a second thermal zone adjacent to the sample holder. The second thermal zone may be angularly separated from the first thermal zone along an axis of rotation of (1) the sample holder or (2) the plurality of thermal zones. The system may also comprise a controller that alternately and sequentially positions the solution in each of the plurality of thermal zones through rotation of the sample holder or the plurality of thermal zones, to conduct the chemical or biological reaction. During the process, (i) in the first thermal zone the solution may be subjected to heating or cooling at a first temperature profile, and (ii) in the second thermal zone the solution may be subjected to heating or cooling at a second temperature profile that is different than the first temperature profile.

In some embodiments, a user may alternately and sequentially position the solution in each of the plurality of thermal zones by directly or indirectly rotating the sample holder or the plurality of thermal zones, to conduct the chemical or biological reaction.

The first thermal zone may comprise a first heating or cooling unit that subjects the solution to heating or cooling at the first temperature profile. The first heating or cooling unit may be a heating unit selected from the group consisting of an infrared (IR) heating unit, a convective heating unit, a Peltier, a resistive heating unit and a heating block. Alternatively, the first heating or cooling unit may be a cooling unit selected from the group consisting of a desiccant, a convective cooling unit and a cooling block.

The second thermal zone may comprise a second heating or cooling unit that subjects the solution to heating or cooling at the second temperature profile. The second heating or cooling unit may be a heating unit selected from the group consisting of an infrared (IR) heating unit, a convective heating unit, a Peltier, a resistive heating unit and a heating block. Alternatively, the second heating or cooling unit may be a cooling unit selected from the group consisting of a desiccant, a convective cooling unit and a cooling block.

The first thermal zone and the second thermal zone may be included on a support. The support may be rotatable with respect to the sample holder.

The chemical or biological reaction may comprise cycling the biological sample between at least two target temperature levels. In the first thermal zone the solution may undergo heating and in the second thermal zone the solution may undergo cooling, or vice versa.

The plurality of thermal zones may further comprise a third thermal zone adjacent to the sample holder. The third thermal zone may be different than the first thermal zone and the second thermal zone. In the third thermal zone, the solution may be subjected to heating or cooling at a third temperature profile. The third temperature profile may be different than the first temperature profile and the second temperature profile. In some embodiments, the third thermal zone may be the same as the first and/or second thermal zone. In some embodiments, the third temperature profile may be the same as the first and/or the second temperature profile.

The first temperature profile may comprise a first target temperature, and the second temperature profile may comprise a second target temperature that is different than the first target temperature. The third temperature profile may comprise a third target temperature.

The controller may comprise one or more computer processors that are individually or collectively programmed to alternately and sequentially position the solution in the first thermal zone and the second thermal zone.

The system may further comprise a detector adjacent to the sample holder. The detector may detect a signal from the solution that is indicative of the chemical or biological reaction or a product of the chemical or biological reaction on the biological sample. The detector may be angularly separated from the first thermal zone and the second thermal zone along the axis of rotation. The controller may position the solution in sensing communication with the detector through rotation of the sample holder or the detector.

In another aspect, the present disclosure provides a method for conducting a chemical or biological reaction on a biological sample. The method may comprise (a) depositing a solution comprising the biological sample in a sample holder. The sample holder may retain the solution during the chemical or biological reaction. The sample holder may be disposed adjacent to a plurality of thermal zones comprising at least a first thermal zone and a second thermal zone. The second thermal zone may be angularly separated from the first thermal zone along an axis of rotation of (1) the sample holder or (2) the plurality of thermal zones. The method may further comprise (b) alternately and sequentially positioning the solution in each of the plurality of thermal zones through rotation of the sample holder or the plurality of thermal zones, to conduct the chemical or biological reaction on the biological sample. During this process, (i) in the first thermal zone the solution may be subjected to heating or cooling at a first temperature profile, and (ii) in the second thermal zone the solution may be subjected to heating or cooling at a second temperature profile that is different than the first temperature profile. Operation (b) may comprise positioning the solution in the first thermal zone and subsequently positioning the solution in the second thermal zone.

The method may further comprise positioning the solution in the first thermal zone subsequent to positioning the solution in the second thermal zone. In some embodiments, the method further comprises positioning the solution in a third thermal zone of the plurality of thermal zones subsequent to positioning the solution in the second thermal zone. The third thermal zone may be different than the first thermal zone and the second thermal zone. In some embodiments, the third thermal zone may be the same as the first and/or second thermal zone.

In some embodiments, operation (b) comprises positioning the solution in the first thermal zone and subsequently positioning the solution in a third thermal zone of the plurality of thermal zones that is different than the second thermal zone. The method may further comprise positioning the solution in the second thermal zone subsequent to positioning the solution in the third thermal zone.

In the first thermal zone, the solution may undergo cooling and in the second thermal zone the solution may undergo heating, or vice versa.

The first temperature profile may comprise a first target temperature. The second temperature profile may comprise a second target temperature that is different than the first target temperature.

In operation (b), the sample holder may rotate the solution from the first thermal zone to the second thermal zone, such that the solution is in thermal communication with the second thermal zone. In some embodiments, in operation (b), the second thermal zone is rotated and brought in thermal communication with the solution.

The method may further comprise positioning the solution in sensing communication with a detector adjacent to the sample holder. The detector may detect a signal from the solution that is indicative of the chemical or biological reaction or a product of the chemical or biological reaction on the biological sample. The detector may be angularly separated from the first thermal zone and the second thermal zone along the axis of rotation. The solution may be positioned in sensing communication with the detector through rotation of the sample holder or the detector.

In any of the various aspects, the sample holder may be rotatable with respect to the first thermal zone and the second thermal zone. The first thermal zone and the second thermal zone may be rotatable with respect to the solution.

In any of the various aspects, the solution may comprise reagents necessary for the chemical or biological reaction. The chemical or biological reaction may be nucleic acid amplification, and the reagents may include one or more primers and polymerizing enzymes. The nucleic acid amplification may be polymerase chain reaction (PCR).

The biological sample may be any suitable sample of a subject. For example, the biological sample may be solid matter (e.g., biological tissue) or may be a fluid (e.g., a biological fluid). In general, a biological fluid can include any fluid associated with living organisms. Non-limiting examples of a biological sample include blood (or components of blood, e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, microbiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues.

The biological sample may be obtained from a subject in a variety of ways. Non-limiting examples of approaches to obtain a biological sample from a subject include accessing the circulatory system (e.g., intravenously or intra-arterially via a syringe or other needle), collecting a secreted biological sample (e.g., feces, urine, sputum, saliva, etc.), surgically (e.g., biopsy), swabbing (e.g., buccal swab, oropharyngeal swab), pipetting, and breathing. Moreover, a biological sample may be obtained from any anatomical part of a subject where a desired biological sample is located.

In some embodiments, the biological sample is from a genome of the subject. In some embodiments, the biological sample is a cell-free nucleic acid sample. For example, the biological sample may be cell-free deoxyribonucleic acid (DNA) or cell-free ribonucleic acid (RNA).

In some embodiments, a biological sample is obtained directly from a subject without further processing. In some embodiments, a biological sample is processed prior to a biological or chemical reaction (e.g., nucleic acid amplification). For example, a lysis agent may be added to a sample holder prior to adding a biological sample and reagents necessary for nucleic acid amplification. Examples of the lysis agent include Tris-HCl, EDTA, detergents (e.g., Triton X-100, SDS), lysozyme, glucolase, proteinase E, viral endolysins, exolysins zymolose, Iyticase, proteinase K, endolysins and exolysins from bacteriophages, endolysins from bacteriophage PM2, endolysins from the B. subtilis bacteriophage PBSX, endolysins from Lactobacillus prophages Lj928, Lj965, bacteriophage 15 Phiadh, endolysin from the Streptococcus pneumoniae bacteriophage Cp-I, bifunctional peptidoglycan lysin of Streptococcus agalactiae bacteriophage B30, endolysins and exolysins from prophage bacteria, endolysins from Listeria bacteriophages, holin-endolysin, cell 20 lysis genes, holWMY Staphylococcus wameri M phage varphiWMY, Iy5WMY of the Staphylococcus wameri M phage varphiWMY, Tween 20, PEG, KOH, NaCl, and combinations thereof. In some embodiments, a lysis agent is sodium hydroxide (NaOH). In some embodiments, the biological sample is not treated with a detergent.

In some embodiments, the biological sample is purified (e.g., by filtration, centrifugation, column purification and/or magnetic purification, for example, by using magnetic beads (e.g., super paramagnetic beads)) to obtain purified nucleic acids.

A cartridge may be used for sample preparation. For example, the cartridge may comprise a filter to separate a sample portion comprising a target nucleic acid from cell debris. In some embodiments, the cartridge comprises reagents for cell lysis (e.g., a lysis buffer or lysis agent as described elsewhere in the present disclosure). In some embodiments, the cartridge is located within and forms a component of the sample holder. In some embodiments, the cartridge is in fluid communication with one or more sample holders, such that samples prepared with the cartridge may flow into the one or more sample holders. A cartridge may be comprised in a system of the present disclosure for conducting a chemical or biological reaction on a biological sample.

A variety of nucleic acid amplification reactions may be used to amplify a target nucleic acid in the biological sample and generate an amplified product. Moreover, amplification of a nucleic acid may be linear, exponential, or a combination thereof. Non-limiting examples of nucleic acid amplification methods include reverse transcription, primer extension, polymerase chain reaction, ligase chain reaction, helicase-dependent amplification (e.g., amplification that is preceded by contacting the nucleic acid with a helicase), asymmetric amplification, rolling circle amplification, and multiple displacement amplification (MDA). In some embodiments, the amplified product may be DNA. In cases where a target RNA is amplified, DNA may be obtained by reverse transcription of the RNA and subsequent amplification of the DNA may be used to generate an amplified DNA product. The amplified DNA product may be indicative of the presence of the target RNA in the biological sample. In cases where DNA is amplified, one or more DNA amplification approaches may be employed. Non-limiting examples of DNA amplification approaches include polymerase chain reaction (PCR), variants of PCR (e.g., real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, touchdown PCR), and ligase chain reaction (LCR). In some embodiments, DNA amplification is linear. In some embodiments, DNA amplification is exponential. In some embodiments, DNA amplification is achieved with nested PCR, which can improve sensitivity of detecting amplified DNA products.

The reagents necessary for nucleic acid amplification may include a polymerizing enzyme and primers having sequence complementary with a target nucleic acid sequence.

In any of the various aspects, primers sets directed to a target nucleic acid may be utilized to conduct nucleic acid amplification reaction. Primer sets generally comprise one or more primers. For example, a primer set may comprise about 1, 2, 3, 4, 5, 6, 7, 8, 9, 10, 20, 30, 40, 50, or more primers. In some embodiments, a primer set comprises primers directed to different amplified products or different nucleic acid amplification reactions. For example, a primer set may comprise a first primer necessary to generate a first strand of nucleic acid product that is complementary to at least a portion of the target nucleic acid and a second primer complementary to the nucleic acid strand product necessary to generate a second strand of nucleic acid product that is complementary to at least a portion of the first strand of nucleic acid product.

For example, a primer set may be directed to a target RNA. The primer set may comprise a first primer that may be used to generate a first strand of nucleic acid product that is complementary to at least a portion the target RNA. In the case of a reverse transcription reaction, the first strand of nucleic acid product may be DNA. The primer set may also comprise a second primer that may be used to generate a second strand of nucleic acid product that is complementary to at least a portion of the first strand of nucleic acid product. In the case of a reverse transcription reaction conducted in parallel with DNA amplification, the second strand of nucleic acid product may be a strand of nucleic acid (e.g., DNA) product that is complementary to a strand of DNA generated from an RNA template.

Where desired, any suitable number of primer sets may be used. For example, about 1, 2, 3, 4, 5, 6, 7, 8, 9, 10, or more primer sets may be used. Where multiple primer sets are used, one or more primer sets may each correspond to a particular nucleic acid amplification reaction or amplified product.

In some embodiments, a DNA polymerase is used. Any suitable DNA polymerase may be used, including commercially available DNA polymerases. A DNA polymerase generally refers to an enzyme that is capable of incorporating nucleotides to a strand of DNA in a template bound fashion. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at a temperature from about 92° C. to 95° C. (e.g., about 94° C. to 95° C.) for a time period from about 2 minutes to 10 minutes may be required, which may change the thermal profile based on different polymerases.

In some embodiments, a reverse transcriptase is used. Any suitable reverse transcriptase may be used. A reverse transcriptase generally refers to an enzyme that is capable of incorporating nucleotides to a strand of DNA, when bound to an RNA template. Non-limiting examples of reverse transcriptases include HIV-1 reverse transcriptase, M-MLV reverse transcriptase, AMV reverse transcriptase, telomerase reverse transcriptase, and variants, modified products and derivatives thereof.

The target nucleic acid sequence may be associated with a disease. The disease may be associated with a virus such as for example an RNA virus or a DNA virus. In some embodiments, the virus may be selected from the group consisting of human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, and Varicella virus. In some embodiments, the influenza virus is selected from the group consisting of H1N1 virus, H3N2 virus, H7N9 virus and H5N1 virus. In some embodiments, the adenovirus is adenovirus type 55 (ADV55) or adenovirus type 7 (ADV7). In some embodiments, the hepatitis C virus is armored RNA-hepatitis C virus (RNA-HCV). In some embodiments, the disease is associated with a pathogenic bacterium (e.g., Mycobacterium tuberculosis) or a pathogenic protozoan (e.g., Plasmodium).

In some embodiments, the disease is cancer. Non-limiting examples of the cancers include colorectal cancer, bladder cancer, ovarian cancer, testicular cancer, breast cancer, skin cancer, lung cancer, pancreatic cancer, stomach cancer, esophageal cancer, brain cancer, leukemia, liver cancer, endometrial cancer, prostate cancer, and head and neck cancer.

The solution may include detectable moieties that permit detection of any signals generated from the biological and/or chemical reactions (e.g., nucleic acid amplification reactions). For example, the detectable moieties may yield a detectable signal whose presence or absence is indicative of a presence of an amplified product. The intensity of the detectable signal may be proportional to the amount of amplified product. In some embodiments, where amplified product is generated of a different type of nucleic acid than the target nucleic acid initially amplified, the intensity of the detectable signal may be proportional to the amount of target nucleic acid initially amplified. For example, in the case of amplifying a target RNA via parallel reverse transcription and amplification of the DNA obtained from reverse transcription, reagents necessary for both reactions may also comprise a detectable moiety that yield a detectable signal indicative of the presence of the amplified DNA product and/or the target RNA amplified. The intensity of the detectable signal may be proportional to the amount of the amplified DNA product and/or the original target RNA amplified. The use of a detectable moiety also enables real-time amplification methods, including real-time PCR for DNA amplification.

Detectable moieties may be linked with nucleic acids, including amplified products, by covalent or non-covalent interactions. Non-limiting examples of non-covalent interactions include ionic interactions, Van der Waals forces, hydrophobic interactions, hydrogen bonding, and combinations thereof. In some embodiments, detectable moieties bind to initial reactants and changes in detectable moiety levels are used to detect amplified product. In some embodiments, detectable moieties are only detectable (or non-detectable) as nucleic acid amplification progresses. In some embodiments, an optically-active dye (e.g., a fluorescent dye) is used as a detectable moiety. Non-limiting examples of dyes include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

In some embodiments, a detectable moiety is a sequence-specific oligonucleotide probe that is optically active when hybridized with an amplified product. Due to sequence-specific binding of the probe to the amplified product, use of oligonucleotide probes can increase specificity and sensitivity of detection. A probe may be linked to any of the optically-active detectable moieties (e.g., dyes) described herein and may also include a quencher capable of blocking the optical activity of an associated dye. Non-limiting examples of probes that may be useful as detectable moieties include TaqMan probes, TaqMan Tamara probes, TaqMan MGB probes, or Lion probes.

In some embodiments and where a detectable moiety is an RNA oligonucleotide probe that includes an optically-active dye (e.g., fluorescent dye) and a quencher positioned adjacently on the probe. The close proximity of the dye with the quencher can block the optical activity of the dye. The probe may bind to a target sequence to be amplified. Upon the breakdown of the probe with the exonuclease activity of a DNA polymerase during amplification, the quencher and dye are separated, and the free dye regains its optical activity that can subsequently be detected.

In some embodiments, a detectable moiety is a molecular beacon. A molecular beacon includes, for example, a quencher linked at one end of an oligonucleotide in a hairpin conformation. At the other end of the oligonucleotide is an optically active dye, such as, for example, a fluorescent dye. In the hairpin configuration, the optically-active dye and quencher are brought in close enough proximity such that the quencher is capable of blocking the optical activity of the dye. Upon hybridizing with amplified product, however, the oligonucleotide assumes a linear conformation and hybridizes with a target sequence on the amplified product. Linearization of the oligonucleotide results in separation of the optically-active dye and quencher, such that the optical activity is restored and may be detected. The sequence specificity of the molecular beacon for a target sequence on the amplified product can improve specificity and sensitivity of detection.

In some embodiments, a detectable moiety is a radioactive species. Non-limiting examples of radioactive species include ¹⁴C, ¹²³I, ¹²⁴I, ¹²⁵I, ¹³¹I, Tc⁹⁹m, ³⁵S, and ³H.

In some embodiments, a detectable moiety is an enzyme that is capable of generating a detectable signal. Detectable signal may be produced by activity of the enzyme with its substrate or a particular substrate in the case the enzyme has multiple substrates. Non-limiting examples of enzymes that may be used as detectable moieties include alkaline phosphatase, horseradish peroxidase, I²-galactosidase, alkaline phosphatase, β-galactosidase, acetylcholinesterase, and luciferase.

The first thermal zone may be an overshooting thermal zone maintained at a heating or cooling overshooting temperature profile (i.e., the first temperature profile). The second thermal zone may be an overshooting thermal zone maintained at a heating or cooling overshooting temperature profile different than the first temperature profile (i.e. the second temperature profile). For example, an overshooting temperature profile (e.g., a heating overshooting temperature profile) may be from about 110° C. to about 140° C., e.g., from about 125° C. to about 135° C. In some embodiments, an overshooting temperature profile (e.g., a heating overshooting temperature profile) may be above or about 110° C., 115° C., 120° C., 125° C., 126° C., 127° C., 128° C., 129° C., 130° C., 131° C., 132° C., 133° C., 134° C., 135° C., 136° C., 137° C., 138° C., 139° C., 140° C., 145° C., or 150° C. An overshooting temperature profile (e.g., a cooling overshooting temperature profile) may be from about 0° C. to about 35° C., e.g., from about 0° C. to about 30° C. In some embodiments, an overshooting temperature profile (e.g., a cooling overshooting temperature profile) may be from about 0° C. to about 20° C. In some embodiments, an overshooting temperature profile (e.g., a cooling overshooting temperature profile) may be from about 5° C. to about 10° C. In some embodiments, an overshooting temperature profile (e.g., a cooling overshooting temperature profile) may be below or about 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., or 35° C.

In some embodiments, the first thermal zone may be a first target thermal zone maintained at a first target temperature profile (i.e., the first temperature profile). The second thermal zone may be a second target thermal zone maintained at a second target temperature profile different than the first temperature profile (i.e. the second temperature profile). For example, a first target temperature profile may be from about 80° C. to about 100° C. For example, a first target temperature profile may be from about 87° C. to about 95° C. A first target temperature profile may be from about 90° C. to about 95° C. A first target temperature profile may be from about 92° C. to about 95° C. A first target temperature profile may be above or about 90° C., 91° C., 92° C., 93° C., 94° C., 95° C., 96° C., 97° C., 98° C., 99° C., or 100° C. A second target temperature profile may be from about 40° C. to about 70° C. A second target temperature profile may be from about 50° C. to about 60° C. A second target temperature profile may be below or about 40° C., 45° C., 50° C., 51° C., 52° C., 53° C., 54° C., 55° C., 56° C., 57° C., 58° C., 59° C., 60° C., 65° C., 70° C., 75° C., 80° C., or 85° C.

In any of the various aspects, the plurality of thermal zones may comprise 1, 2, 3, 4, 5, 6, 7, 8, 9, 10, or more thermal zones. For example, the plurality of thermal zones may comprise 1, 2, 3, 4, 5, 6, 7, 8, 9, 10, or more target thermal zones, and/or may comprise 1, 2, 3, 4, 5, 6, 7, 8, 9, 10, or more overshooting thermal zones.

In some embodiments, the plurality of thermal zones may comprise a first target thermal zone held at a first target temperature profile from about 92° C. to about 95° C., a first overshooting thermal zone held at a first overshooting temperature profile from about 110° C. to about 140° C., a second target thermal zone held at a second target temperature profile from about 40° C. to about 70° C., and a second overshooting thermal zone held at a second overshooting temperature profile from about 0° C. to about 20° C. In some embodiments, the plurality of thermal zones may comprise a first target thermal zone held at a first target temperature profile of at least about 95° C., a first overshooting thermal zone held at a first overshooting temperature profile of at least about 135° C., a second target thermal zone held at a second target temperature profile of at least about 55° C., and a second overshooting thermal zone held at a second overshooting temperature profile of below or about 8° C.

In some embodiments, the plurality of thermal zones may comprise a first overshooting thermal zone held at a first overshooting temperature profile from about 110° C. to about 140° C., and a second overshooting thermal zone held at a second overshooting temperature profile from about 0° C. to about 20° C. In some embodiments, the plurality of thermal zones may comprise a first overshooting thermal zone held at a first overshooting temperature profile of at least about 135° C., and a second overshooting thermal zone held at a second overshooting temperature profile of below or about 8° C.

A thermal zone may remain set to a particular temperature profile throughout an entire reaction process. Alternatively, a thermal zone may be changed from one temperature profile to another during a reaction process. A thermal zone may be set to 1, 2, 3, 4, 5, or more different temperature levels during a reaction process.

A thermal zone may comprise indentations, slots, holes, depressions, or other shapes designed to mate with sample holders. Such designs may provide improved thermal contact between the thermal zone and the sample holder. In some embodiments, the thermal zones may be flat. In some embodiments, a sample holder may comprise a flat surface to contact with a flat surface of a thermal zone.

Thermal zones may be brought into thermal contact with sample volumes (e.g., reaction vessels) through a variety of motions. Thermal zones may be moved into contact with sample volumes, or sample volumes may be moved into contact with thermal zones. In some embodiments, thermal zones may be mounted on or otherwise coupled to moveable elements, such as arms (e.g. linear arms, rotating arms), belts, cams, discs, levers, tracks, or wheels. Such moveable elements may be driven by one or more motors, springs, or other driving elements. In some embodiments, sample volumes (e.g., in sample holders or reaction vessels) may be mounted on or otherwise coupled to moveable elements, such as arms (e.g. linear arms, rotating arms), belts, cams, discs, levers, tracks, or wheels. Such moveable elements may be driven by one or more motors, springs, or other driving elements. Moveable elements may be coupled or linked to coordinate movement.

Movement of moveable elements may be controlled by a timing control system. The timing control system may be electronic or mechanical. An electronic timing control system may comprise one or more computer processors. The electronic timing control system may be operated to move the thermal zones into and out of thermal contact with the sample holder and/or sample volumes in a determined order and for determined amounts of time. In some embodiments, the timing control system may be mechanical. For example, the thermal zones and/or the sample holder may be mounted on moveable elements, and these moveable elements may be connected to a mechanical timing control system such as a belt or cam. The moveable elements may be connected to the mechanical timing control system such that, when the mechanical timing control system is operated, the moveable elements move the thermal zones into and out of thermal contact with sample volumes in a determined order and for determined amounts of time.

In some embodiments, the moveable elements may be operated directly or indirectly by a user. For example, a user may manually control movement of the moveable elements to alternately and sequentially position the sample holder or sample volume in each of the plurality of thermal zones.

The amount of time each thermal zone is placed in thermal contact with a sample volume may be the same or different for the plurality of thermal zones. A thermal zone may be in thermal contact with a sample volume for at least about 0.1, 0.2, 0.3, 0.4, 0.5, 0.6, 0.7, 0.7, 0.8, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 35, 40, 45, 50, or 55 seconds, or for about 1, 1.5, 2, 2.5, 3, 3.5, 4, 4.5, or 5 minutes or more.

Movements can follow any suitable path, including but not limited to linear, curved, and sinusoidal. In some examples, a curving path for a movement can provide simpler and faster actuation than that provided by a linear motion. In some examples, a curving path for movement can reduce or eliminate the need for high-precision control. In some examples, a curving path for movement can reduce the volume of the device. In some embodiments, the sample holder remains stationary while thermal zones move in and out of thermal contact with the sample holder. In some embodiments, the thermal zones remain stationary while the sample holder moves in and out of thermal contact with the thermal zones. In some embodiments, both the sample holder and the thermal zones move to bring the sample holder into or out of thermal contact with one or more thermal zones.

In some embodiments, the plurality of thermal zones are angularly separated from each other along an axis of movement (e.g., rotation) of the sample holder or the plurality of thermal zones. For example, the plurality of thermal zones are separated from each other by at least 1°, 5°, 10°, 15°, 20°, 25°, 30°, 35°, 40°, 45°, 50°, 55°, 60°, 65°, 70°, 75°, 80°, 85°, 90°, 100°, 105°, 110°, 120°, 130°, 140°, 150°, 160°, 170°, 180°, 190°, 200°, 210°, 220°, 230°, 240° or more along an axis of movement (e.g., rotation) of the sample holder or the plurality of thermal zones.

The sample holder may comprise a reaction vessel (e.g., a PCR tube) that receives a solution comprising a biological sample. The reaction vessel may be of varied size, shape, weight, and configuration. In some embodiments, the reaction vessel is round or oval tubular shaped. In some embodiments, the reaction vessel is rectangular, square, diamond, circular, elliptical, or triangular shaped. The reaction vessel may be regularly shaped or irregularly shaped. For example, a reaction vessel may be a tube, a well, a capillary tube, a cartridge, a cuvette, a centrifuge tube, or a pipette tip. In some embodiments, the reaction vessel has a surface area to volume ratio of at least 100 mm⁻¹, 200 mm⁻¹, 300 mm⁻¹, 350 mm⁻¹, 400 mm⁻¹, 450 mm⁻¹, 500 mm⁻¹, 1×10³ mm⁻¹, 1×10⁴ mm⁻¹, 1×10⁵ mm⁻¹, 1×10⁶ mm⁻¹, 1×10⁷ mm⁻¹, 1×10⁸ mm⁻¹, 1×10⁹ mm⁻¹, 1×10¹⁰ mm⁻¹, 1×10¹¹ mm⁻¹, 1×10¹² mm⁻¹, 1×10¹³ mm⁻¹, 1×10¹⁴ mm⁻¹, 1×10¹⁵ mm⁻¹ or more.

In some embodiments, a reaction vessel is part of an array of reaction vessels. An array of reaction vessels may be used for automating methods and/or simultaneously processing multiple samples. For example, a reaction vessel may be a well of a microwell plate comprised of a number of wells. An array of reaction vessels may comprise any appropriate number of reaction vessels. For example, an array may comprise at least 2, 4, 6, 8, 10, 12, 14, 16, 18, 20, 25, 35, 48, 96, 144, 384, or more reaction vessels. A reaction vessel part of an array of reaction vessels may also be individually addressable by a fluid handling device, such that the fluid handling device can correctly identify a reaction vessel and dispense appropriate fluid materials into the reaction vessel. Fluid handling devices may be useful in automating the addition of fluid materials to reaction vessels.

In any of the various aspects, a source of excitation energy (e.g., a light-emitting diode, a laser or other energy sources) may be activated to direct excitation energy to the solution comprising the biological sample, thereby generating emitted signals (e.g., optical signals, fluorescent signals and/or electrostatic signals) indicating occurrence and/or result of the chemical or biological reaction on the biological sample. The signals generated may then be detected with a detector (e.g., a charge-coupled device camera). The detector may be positioned adjacent to the sample holder. In some embodiments, the detector is angularly separated from the plurality of thermal zones along an axis of rotation of the sample holder or the plurality of thermal zones.

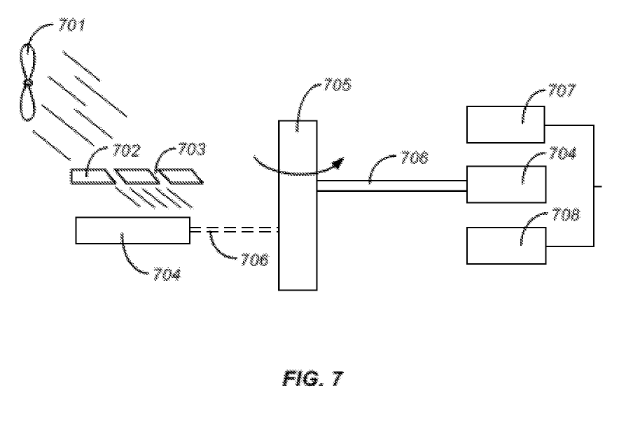

FIG. 7 provides an example of a system for conducting a chemical or biological reaction on a biological sample. A sample holder 704 (e.g., a PCR reaction vessel) may be mounted to a support 706 (e.g., a rotatable arm or a rotatable flat disc) at an end thereof (e.g., when the support 706 is a rotatable arm) or near a peripheral region distant from the center thereof (when the support 706 is a rotatable flat disc). A controller 705 driven by a motor may be connected to the support 706 at an end opposite to the sample holder 704 (e.g., when the support 706 is a rotatable arm) or in the center thereof (when the support 706 is a rotatable flat disc). The system further comprises a first thermal zone composed of a first heating block 707 and a second heating block 708. In addition, the system comprises a second thermal zone composed of cooling blocks 702 (e.g., metal blocks) and a fluid flow unit 701 (e.g., a fan). Rotation of the controller 705 may subject the sample holder 704 to rotation, thereby alternately and sequentially positioning the sample holder 704 (and a reaction solution comprising a biological sample therein) in the first thermal zone (e.g., between the first heating block 707 and the second heating block 708) and the second thermal zone (e.g., below the cooling blocks 702). The heating blocks 707 and 708 may be in thermal communication with the sample holder 704, thereby raising the temperature of the solution within the sample holder 704. The cooling blocks 702 may be arranged in a way such that when the sample holder 704 is rotated into the second thermal zone (e.g., to a position below the cooling blocks 702), a cooling fluid (e.g., cool air) may be allowed to flow from the fluid flow unit 701 through spaces 703 (e.g., pores) located between two neighboring cooling blocks 702 towards the sample holder 704, thereby lowering the temperature of the solution within the sample holder 704.

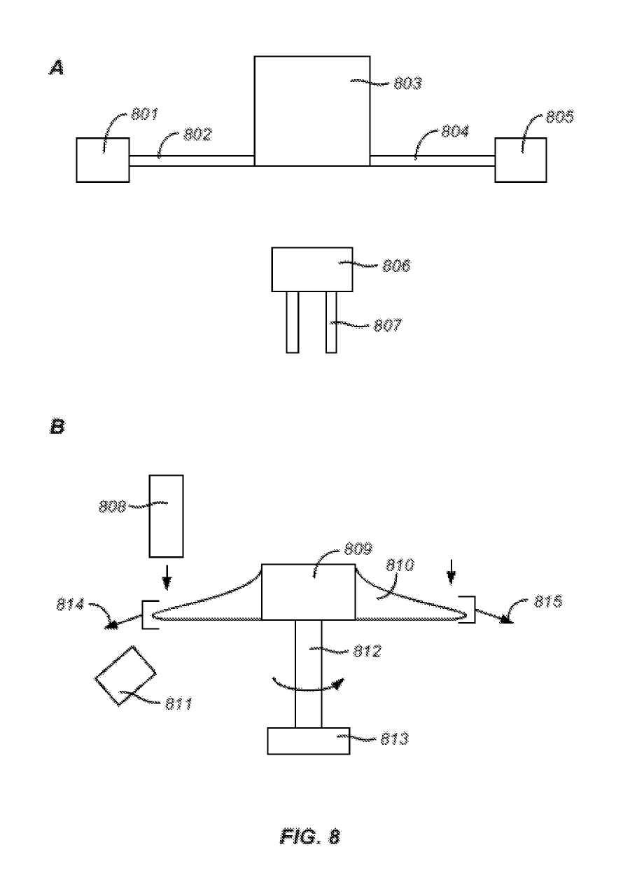

FIG. 8 (panel A) illustrates a sample preparation assembly comprising a cartridge 803 for sample processing and preparation, the cartridge 803 may be in fluid communication with a first sample holder 801 and a second sample holder 805 through a first channel 802 and a second channel 804, respectively. The first sample holder 801 and the second sample holder 805 may be identical or different. The cartridge 803 may be coupled with a motor 806 via one or more engaging elements 807 (e.g. shafts) connected to the motor 806. The motor 806 may drive rotation of the sample holders 801 and 805 that are in fluid communication with the cartridge 803.

FIG. 8 (panel B) provides an example of a system for conducting a chemical or biological reaction on a biological sample. The system may comprise a cartridge 809 for sample preparation, the cartridge 809 may be in fluid communication with one or more sample holders 810 comprising the biological sample. Activation of a motor 813 may drive rotation of a controller 812 connected to the cartridge 809, thereby alternately and sequentially positioning the one or more sample holder 810 in a heating thermal zone 815 and a cooling thermal zone 814. In addition, the system may comprise an excitation energy source 808 located adjacent to and above the plane of rotation of the sample holder 810 and a detector 811 located adjacent to and below the plane of rotation of the sample holder 810. Activation of the excitation energy source 808 may direct energy to the solution in the sample holder 810, thereby generating a signal that may be detected with the detector 811.

### Fluidic Cooling

In another aspect, the present disclosure provides an apparatus for cooling a solution comprising a biological sample (e.g., nucleic acid sample) during a chemical or biological reaction (e.g., a nucleic acid amplification reaction). The apparatus may comprise a first chamber comprising a heat transfer material having a phase transition temperature in a range of about −100° C. to 50° C. In some embodiments, the first chamber comprises a heat transfer material having a phase transition temperature of at least about −120° C., −110° C., −100° C., −95° C., −90° C., −85° C., −80° C., −75° C., −70° C., −65° C., −60° C., −55° C., −50° C., to about 0° C., 10° C., 20° C., 30° C., 40° C., or 50° C., such as, e.g., about −120° C. to 20° C., −110° C. to 30° C., −100° C. to 50° C., −100° C. to 60° C., −100° C. to 70° C., −95° C. to 55° C., −90° C. to 50° C., etc. The apparatus may further comprise a second chamber comprising a substrate having a heat transfer surface. The second chamber may be fluidically isolated from the first chamber, and the heat transfer surface may be in thermal communication with the solution comprising the biological sample during the chemical or biological reaction. The apparatus may further comprise a control unit that brings the second chamber in fluid communication with the first chamber in accordance with a timing that at least partially depends upon a duration of the chemical or biological reaction. When the second chamber is in fluid communication with the first chamber, the heat transfer material may undergo a phase transition that draws thermal energy from the substrate along the heat transfer surface to subject the solution to cooling.

In another aspect, the present disclosure provides a method for cooling a solution comprising a biological sample (e.g., a nucleic acid sample) during a chemical or biological reaction (e.g., a nucleic acid amplification reaction). The method may comprise (a) activating a heat exchange apparatus comprising (1) a first chamber comprising a heat transfer material having a phase transition temperature in a range of about −100° C. to 50° C.; and (2) a second chamber comprising a substrate having a heat transfer surface. In some embodiments, the first chamber comprises a heat transfer material having a phase transition temperature of at least about −120° C., −110° C., −100° C., −95° C., −90° C., −85° C., −80° C., −75° C., −70° C., −65° C., −60° C., −55° C., 50° C., to about 0° C., 10° C., 20° C., 30° C., 40° C., or 50° C., such as, e.g., about −120° C. to 20° C., −110° C. to 30° C., −100° C. to 50° C., −100° C. to 60° C., −100° C. to 70° C., −95° C. to 55° C., −90° C. to 50° C., etc. The second chamber may be fluidically isolated from the first chamber, and the heat transfer surface may be in thermal communication with a solution comprising the biological sample during the chemical or biological reaction. The method may further comprise (b) bringing the second chamber in fluid communication with the first chamber in accordance with a timing that at least partially depends upon a duration of the chemical or biological reaction. When the second chamber is in fluid communication with the first chamber, the heat transfer material may undergo a phase transition that draws thermal energy from the substrate along the heat transfer surface to subject the solution to cooling. The method may further comprise (c) subjecting the solution to cooling using the thermal energy drawn from the substrate along the heat transfer surface.

In any of the various aspects, the heat transfer surface may be in thermal communication with the solution indirectly through at least one heat transfer medium. The at least one heat transfer medium may be a cooling fluid. In some embodiments, the heat transfer surface is in thermal communication with the solution directly.

In any of the various aspects, the apparatus may further comprise a seal between the first chamber and the second chamber, and the seal may (i) isolate the second chamber from the first chamber when in a closed configuration, and (ii) bring the second chamber in fluid communication with the first chamber when in an open configuration. During use, (i) the seal may be actuated from the closed configuration to the open configuration to bring the first chamber in fluid communication with the second chamber, and (ii) the heat transfer material may undergo a phase transition, the phase transition may draw thermal energy from the substrate along the heat transfer surface to subject the solution to cooling. For example, bringing the second chamber in fluid communication with the first chamber may comprise actuating the seal from the closed configuration to the open configuration to bring the first chamber in fluid communication with the second chamber, when the seal is in an open configuration, the heat transfer material may undergo a phase transition that draws thermal energy from the substrate along the heat transfer surface to subject the solution to cooling. The seal may be part of a fluid flow path between the first chamber and the second chamber. In some embodiments, the seal is actuated from the closed configuration to the opening configuration by piercing. The seal may be part of a valve between the first chamber and the second chamber, and the seal may be actuated from the closed configuration to the open configuration by opening the valve.

In some embodiments, during use, the heat transfer material is subjected to flow from the first chamber to the second chamber to come in contact with the heat transfer surface, and upon contact with the heat transfer surface, the heat transfer material may undergo the phase transition to yield a vapor. The phase transition may draw thermal energy from the substrate along the heat transfer surface to subject the solution to cooling. For example, bringing the second chamber in fluid communication with the first chamber may comprise subjecting the heat transfer material to flow from the first chamber to the second chamber to come in contact with the heat transfer surface, upon contact with the heat transfer surface, the heat transfer material may undergo the phase transition to yield a vapor.

The heat transfer material may be a heat transfer liquid. During use, the heat transfer liquid may be subjected to flow from the first chamber to the second chamber to come in contact with the heat transfer surface. For example, bringing the second chamber in fluid communication with the first chamber may comprise subjecting the heat transfer liquid to flow from the first chamber to the second chamber to come in contact with the heat transfer surface. The heat transfer liquid may be water. In some embodiments, the heat transfer liquid may comprise an alcohol. The alcohol may be isopropyl alcohol, methanol, ethanol, propanol, butanol or pentanol.

The apparatus may further comprise a third chamber in fluid communication with the second chamber through at least one fluid flow path between the second chamber and the third chamber, the third chamber may receive a vapor generated upon the heat transfer material undergoing the phase transition. The third chamber may comprise a capture material that captures the vapor. The capture material may be a hygroscopic substance. For example, the capture material may be a desiccant.

The heat transfer material may be selected, at least in part, on the basis of its vapor pressure, such that the heat transfer material is sufficiently volatile in the second chamber. For example, in some cases, the vapor pressure of the heat transfer material may be at least about 0.5 kilopascals (kPa), 1.0 kPa, 1.5 kPa, 2.0 kPa, 2.5 kPa, 3.0 kPa, 3.5 kPa, 4.0 kPa, 4.5 kPa, 5.0 kPa, 5.5 kPa, 6.0 kPa, 6.5 kPa, 7.0 kPa, 7.5 kPa, 8.0 kPa, 8.5 kPa, 9.0 kPa, 9.5 kPa, 10.0 kPa, 10.5 kPa, 11.0 kPa, 11.5 kPa, 12.0 kPa, 12.5 kPa, 13.0 kPa, 13.5 kPa, 14.0 kPa, 14.5 kPa, 15.0 kPa or more. In some cases, the vapor pressure of the heat transfer material may be at most about 15.0 kPa, 14.5 kPa, 14.0 kPa, 13.5 kPa, 13.0 kPa, 12.5 kPa, 12.0 kPa, 11.5 kPa, 11.0 kPa, 10.5 kPa, 10.0 kPa, 9.5 kPa, 9.0 kPa, 8.5 kPa, 8.0 kPa, 7.5 kPa, 7.0 kPa, 6.5 kPa, 6.0 kPa, 5.5 kPa, 5.0 kPa, 4.5 kPa, 4.0 kPa, 3.5 kPa, 3.0 kPa, 2.5 kPa, 2.0 kPa, 1.5 kPa, 1.0 kPa, 0.5 kPa or less.

Moreover, in some cases, the heat transfer material may comprise molecules that have a carbon backbone. In such cases, the heat transfer material can be selected, at least in part, on the basis of the number of carbon atoms in its carbon backbone. For example, the carbon backbone of a heat transfer material can comprise at least 1 carbon atom, at least 2 carbon atoms, at least 3 carbon atoms, at least 4 carbon atoms, at least 5 carbon atoms, at least 6 carbon atoms, at least 7 carbon atoms, at least 8 carbon atoms, at least 9 carbon atoms, at least 10 carbon atoms or more. In some cases, the carbon backbone of a heat transfer material comprises at most 10 carbon atoms, at most 9 carbon atoms, at most 8 carbon atoms, at most 7 carbon atoms, at most 6 carbon atoms, at most 5 carbon atoms, at most 4 carbon atoms, at most 3 carbon atoms, at most 2 carbon atoms or at most 1 carbon atom.

In a method of the present disclosure, activating the heat exchange apparatus may comprise providing the capture material in the third chamber prior to, bringing the second chamber in fluid communication with the first chamber. The third chamber may be in fluid communication with a pump that draws the vapor. The third chamber may be in fluid communication with a fluid flow unit that subjects the vapor to flow from the third chamber to a vapor repository. The fluid flow unit may be a fan, a compressor, and/or a pump.

In any of the various aspects, the substrate may comprise an additional heat transfer surface. The method may further comprise bringing a cooling fluid in contact with the additional heat transfer surface to subject the cooling fluid to cooling. The cooling fluid may comprise water or an alcohol. The method may further comprise using the cooling fluid to cool a reaction tube comprising the solution.

In any of the various aspects, the chemical or biological reaction may be nucleic acid amplification.

In a method of the present disclosure, activating the heat exchange apparatus may comprise providing the heat transfer material in the first chamber. In some embodiments, activating the heat exchange apparatus comprises bringing the second chamber in fluid communication with the first chamber.

In another aspect, the present disclosure provides an apparatus for cooling a solution comprising a biological sample (e.g., nucleic acid sample) during a chemical or biological reaction (e.g., nucleic acid amplification reaction). The apparatus may comprise a first chamber comprising a heat transfer material. The apparatus may also comprise a second chamber comprising a substrate having a heat transfer surface. The second chamber may be fluidically isolated from the first chamber, and the heat transfer surface may be in thermal communication with the solution comprising the biological sample during the chemical or biological reaction. The apparatus may further comprise a seal between the first chamber and the second chamber, and the seal may (i) isolate the second chamber from the first chamber when in a closed configuration, and (ii) bring the second chamber in fluid communication with the first chamber when in an open configuration. During use, the seal may be actuated from the closed configuration to the open configuration to bring the first chamber in fluid communication with the second chamber. In the open configuration, the heat transfer material may undergo a phase transition that draws thermal energy from the substrate along the heat transfer surface to subject the solution to cooling.

In a further aspect, the present disclosure provides a method for cooling a solution comprising a biological sample (e.g., nucleic acid sample) during a chemical or biological reaction (e.g., nucleic acid amplification reaction). The method may comprise (a) activating a heat exchange apparatus comprising (1) a first chamber comprising a heat transfer material; (2) a second chamber comprising a substrate having a heat transfer surface. The second chamber may be fluidically isolated from the first chamber, and the heat transfer surface may be in thermal communication with the solution comprising the biological sample during the chemical or biological reaction. The heat exchange apparatus may further comprise (3) a seal between the first chamber and the second chamber, the seal may (i) isolate the second chamber from the first chamber when in a closed configuration, and (ii) bring the second chamber in fluid communication with the first chamber when in an open configuration The method may also comprise (b) actuating the seal from the closed configuration to the open configuration to bring the first chamber in fluid communication with the second chamber. In the open configuration, the heat transfer material may undergo a phase transition that draws thermal energy from the substrate along the heat transfer surface to subject the solution to cooling.

In any of the various aspects, the heat transfer surface may be in thermal communication with the solution indirectly through at least one heat transfer medium. The heat transfer surface may be in thermal communication with the solution directly. The heat transfer material may be a heat transfer liquid.

Bringing the first chamber in fluid communication with the second chamber may comprise subjecting the heat transfer liquid to flow from the first chamber to the second chamber to come in contact with the heat transfer surface.

In any of the various aspects, the apparatus may further comprise a third chamber in fluid communication with the second chamber through at least one fluid flow path between the second chamber and the third chamber. The third chamber may receive a vapor generated upon the heat transfer material undergoing the phase transition. The third chamber may comprise a capture material that captures the vapor. The capture material may be a hygroscopic substance. For example, the capture material may be a desiccant.

Activating the heat exchange apparatus may comprise providing the capture material in the third chamber prior to bringing the first chamber in fluid communication with the second chamber. The substrate may comprise an additional heat transfer surface.

A method of the present disclosure may further comprise bringing a cooling fluid in contact with the additional heat transfer surface to subject the cooling fluid to cooling. The method may further comprise using the cooling fluid to cool a reaction tube comprising the solution.

A method of the present disclosure may further comprise (c) subjecting the solution to cooling using the thermal energy drawn from the substrate along the heat transfer surface.

In any of the various aspects, the chemical or biological reaction may be nucleic acid amplification.

Activating a heat exchange apparatus may comprise providing the heat transfer material in the first chamber. In some embodiments, activating a heat exchange apparatus comprises bringing the second chamber in fluid communication with the first chamber.

The third chamber may be in fluid communication with a pump that draws the vapor. The third chamber may be in fluid communication with a fluid flow unit that subjects the vapor to flow from the third chamber to a vapor repository. The fluid flow unit may be a fan, a compressor, and/or a pump.

In any of the various aspects, the substrate may comprise an additional heat transfer surface, and during use, a cooling fluid may be brought in contact with the additional heat transfer surface to subject the cooling fluid to cooling.

In any of the various aspects, the control unit may comprise one or more computer processors that are individually or collectively programmed to bring the second chamber in fluid communication with the first chamber in accordance with the timing.

In any of the various aspects, the transfer surface may be in thermal communication with the solution indirectly through at least one heat transfer medium. The at least one heat transfer medium may be a cooling fluid. The heat transfer surface may be in thermal communication with the solution directly.

During use, the heat transfer material may be subjected to flow from the first chamber to the second chamber to come in contact with the heat transfer surface, and upon contact with the heat transfer surface, the heat transfer material may undergo the phase transition that draws thermal energy from the substrate along the heat transfer surface.

In any of the various aspects, the seal may be part of a fluid flow path between the first chamber and the second chamber. The seal may be actuated from the closed configuration to the opening configuration by piercing. The seal may be part of a valve between the first chamber and the second chamber, and the seal may be actuated from the closed configuration to the open configuration by opening the valve.

The heat transfer material may be a heat transfer liquid. In some examples, the heat transfer material is an alcohol, aldehyde, ketone, or carboxylic acid. For example, the heat transfer material is an alcohol, such as isopropyl alcohol. As an alternative, the heat transfer material may be a heat transfer gas, such as air.

A heat transfer surface may have any suitable shape or configuration. In some embodiments, the heat transfer surface is a flat surface. In some embodiments, the heat transfer surface is curved. The heat transfer surface may be made of or coated with any suitable material (e.g., a heat transfer material). For example, the heat transfer surface may be made of or coated with a material with a heat capacity of at least about 0.2 J/g*K, at least about 0.3 J/g*K, at least about 0.4 J/g*K, at least about 0.5 J/g*K, at least about 1.0 J/g*K or more. Moreover, the heat transfer surface may comprise or may be coated with a material that comprises any suitable specific heat. For example, the specific heat of a heat transfer surface material or heat transfer surface coating material may be at least about 0.001 calories/gram (cal/g), at least about 0.005 cal/g, at least about 0.01 cal/g, at least about 0.05 cal/g, at least about 0.1 cal/g, at least about 0.5 cal/g, at least about 1.0 cal/g, 1 least about 1.5 cal/g, at least about 2.0 cal/g, at least about 2.5 cal/g, at least about 3.0 cal/g, at least about 3.5 cal/g, at least about 4.0 cal/g, at least about 4.5 cal/g, at least about 5.0 cal/g, at least about 5.5 cal/g, at least about 6.0 cal/g, at least about 6.5 cal/g, at least about 7.0 cal/g, at least about 7.5 cal/g, at least about 8.0 cal/g, at least about 8.5 cal/g, at least about 9.0 cal/g, at least about 9.5 cal/g, at least about 10.0 cal/g or more. In some cases, the specific heat of a heat transfer surface material or heat transfer surface coating material is at most about 10.0 cal/g, at most about 9.5 cal/g, at most about 9.0 cal/g, at most about 8.5 cal/g, at most about 8.0 cal/g, at most about 7.5 cal/g, at most about 7.0 cal/g, at most about 6.5 cal/g, at most about 6.0 cal/g, at most about 5.5 cal/g, at most about 5.0 cal/g, at most about 4.5 cal/g, at most about 4.0 cal/g, at most about 3.5 cal/g, at most about 3.0 cal/g, at most about 2.5 cal/g, at most about 2.0 cal/g, at most about 1.5 cal/g, at most about 1.0 cal/g, at most about 0.5 cal/g, at most about 0.1 cal/g, at most about 0.05 cal/g, at most about 0.01 cal/g, at most about 0.005 cal/g, at most about 0.001 cal/g, or less.

In some embodiments, the heat transfer surface is made of or coated with a metal or a metal alloy. For example, the metal or metal alloy may comprise nickel, copper, chromium or their alloys. In some embodiments, the metal or metal alloy comprises silver, chlorinated polymers and/or fluorinated polymers. In some embodiments, the heat transfer surface comprises a polymer comprising a metal. In some embodiments, the heat transfer surface comprises carbon (e.g., graphene). In some embodiments, the heat transfer material may have a phase transition temperature in a range of at least about −120° C., −110° C., −100° C., −95° C., −90° C., −85° C., −80° C., −75° C., −70° C., −65° C., −60° C., −55° C., −50° C., to about 0° C., 10° C., 20° C., 30° C., 40° C., or 50° C., such as, e.g., about −120° C. to 20° C., −110° C. to 30° C., −100° C. to 50° C., −100° C. to 60° C., −100° C. to 70° C., −95° C. to 55° C., −90° C. to 50° C., etc.

A seal may be of any suitable structure that separates at least two volumes, or that separates a volume from its external environment. A seal may be made of or may comprise a synthetic membrane, e.g., a membrane formed of a solid state material (e.g., semiconductor, metal, semi-metal or non-metal) or polymeric material (e.g., a polymeric membrane). For example, a seal may be formed by an opaque, transparent, or translucent material separating the first chamber from the second chamber. In some embodiments, the seal is a polymeric membrane made from parafilm.

A capture material may be any suitable material capable of capturing a vapor and/or a liquid. For example, the capture material may be made of or comprise a hygroscopic substance. The hygroscopic substance may comprise any substance capable of attracting and/or holding water molecules from the surrounding environment, such as cellulose fibers, sugar, glycerol, ethanol, methanol, sulfuric acid, salts, or a combination thereof. In some embodiments, the capture material is a desiccant.

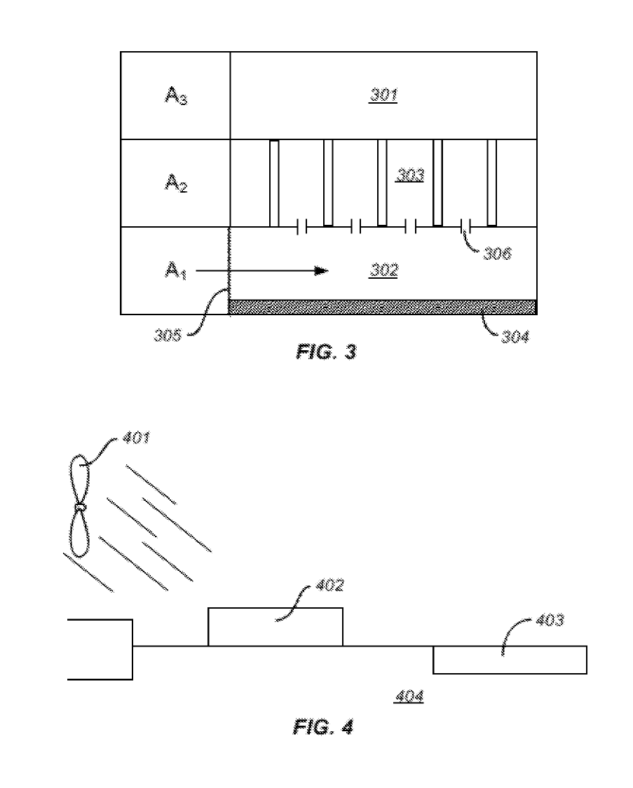

FIG. 3 provides an example of the apparatus for heat exchange. The apparatus includes first chambers A1, A2 and A3. One or more of the first chambers, A1, A2 and A3 may be fluidically connected (e.g., via membranes between chambers or channels between chambers) with one or both of the other first chambers or one or more of the first chambers A1, A2 and A3 may be isolated (e.g., via chamber walls) from one or both of the other first chambers. In some embodiments, heat transfer fluids from one or more of first chambers A1, A2, and A3 are mixed together to form a heat transfer fluid mixture. In other embodiments, each of the first chambers A1, A2, and A3 may each provide a separate heat transfer fluid that may be used concurrently with a heat transfer fluid provided from another first chamber or in a separate cooling cycle process. The apparatus may further comprise a second chamber 302. The second chamber 302 may be separated from one or more of the first chambers A1, A2 and A3 by a penetrable seal 305 associated with a respective first chamber and may comprise a substrate having a heat transfer surface 304. In addition, the apparatus may comprise a third chamber 301 that is in fluid communication with the second chamber 302 through a plurality of fluid flow paths 303. Each of the plurality of fluid flow paths 303 may comprise an opening 306 in an end thereof adjacent to the second chamber 302.

During use, a seal 305 associated with a given first chamber may be actuated (e.g., be pierced or removed by a user) to an open configuration, so that its heat transfer fluid is released from the given first chamber into the second chamber 302 and come in contact with the heat transfer surface 304. Upon contact with the heat transfer surface 304, the heat transfer fluid may undergo evaporation to yield a vapor that flows via the openings 306 through the plurality of fluid flow paths 303 to the third chamber 301. The evaporation may draw thermal energy from the substrate along the heat transfer surface 304 to subject the substrate to cooling. The third chamber 301 may comprise a capture material (e.g., a desiccant) that captures the vapor.

FIG. 4 provides an example of the apparatus for heat exchange that may be employed in nucleic acid amplification (the nucleic acid amplification is as described elsewhere in the present disclosure). A fluid flow unit (e.g., a fan) 401 may be activated to generate a fluid flow that may flow through a fluid flow path 404 adjacent to a cooling apparatus 402 (e.g. an apparatus as demonstrated in FIG. 3), thereby generating a cooled fluid (e.g., cooled air) that may be used to lower the temperature of a sample holder 403 (e.g., a PCR vessel) located at one end of the fluid flow path 404.

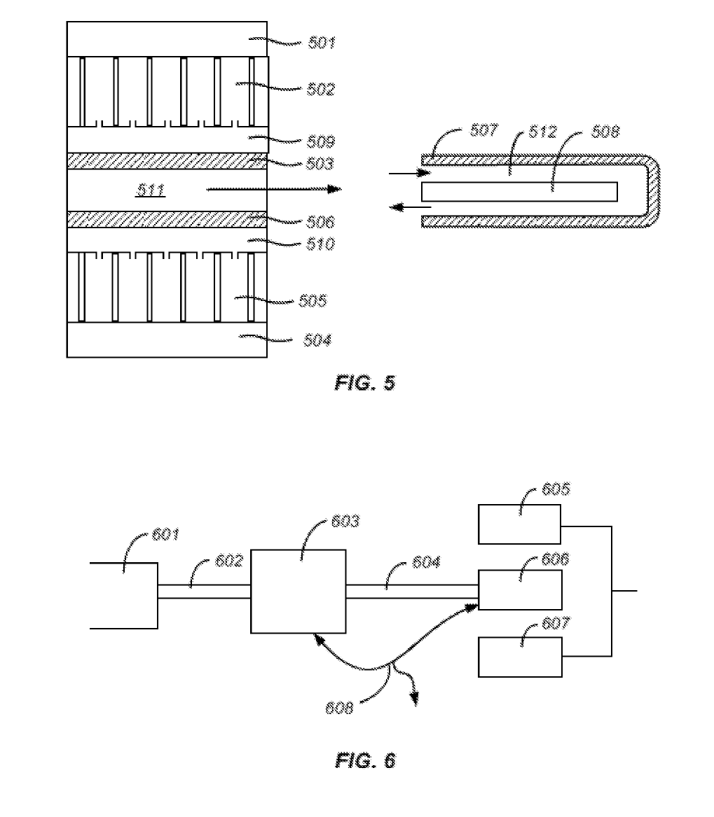

FIG. 5 provides another example of the apparatus for heat exchange that may be employed in nucleic acid amplification (the nucleic acid amplification is as described elsewhere in the present disclosure). A cooling liquid may flow through a fluid flow path 511 formed between two cooling apparatus (e.g. an apparatus as demonstrated in FIG. 3). Each cooling apparatus may comprise a chamber 509 or 510 containing a substrate with a heat transfer surface 503 or 506. A heat transfer liquid may be released into the chamber 509 or 510 and come in contact with the heat transfer surface 503 or 506, respectively. Upon contact with the heat transfer surface 503 or 506, the heat transfer liquid may undergo evaporation to yield a vapor that flows through a plurality of fluid flow paths 502 or 505, respectively, to a different chamber 501 or 504. The evaporation may draw thermal energy from the substrate along the heat transfer surface 503 or 506 to subject the substrate to cooling. The cooling liquid flowing through the fluid flow path 511 may be subjected to cooling by the cooled subjects of the cooling apparatus. The cooled cooling liquid may flow from the fluid flow path 511 into a U-shaped flow path 512. The flow path 512 is formed between a U-shaped boundary 507 and walls of a PCR vessel 508, thereby surrounding the PCR vessel 508 and cooling the samples therein.

FIG. 6 provides an example of the apparatus for heat exchange that may be employed in combination with a system for conducting a chemical or biological reaction on a biological sample. A cooling fluid (e.g., water) may be released with a time-controlled approach from a reservoir 601 that is in fluid communication with a cooling apparatus 603 through a fluid flow path 602. The cooling fluid may be cooled down after passing across the cooling apparatus 603, as described elsewhere in the present disclosure. A sample holder (e.g., a PCR vessel) 606 may be positioned between a first heating block 605 and a second heating block 607, and the cooling fluid may be coupled to the sample holder 606 via a flow path 604 or other fluidic channels 608. When the heating blocks 605 and 607 are activated, the temperature of solutions within the sample holder 606 may be elevated, and when a target temperature is reached and cooling is desired, the heating blocks 605 and 607 are deactivated, and the cooling fluid is released to flow across the cooling apparatus towards the sample holder 606 to lower the temperature of solutions therein. The heating and cooling processes may be repeated as necessary.

### Control Systems

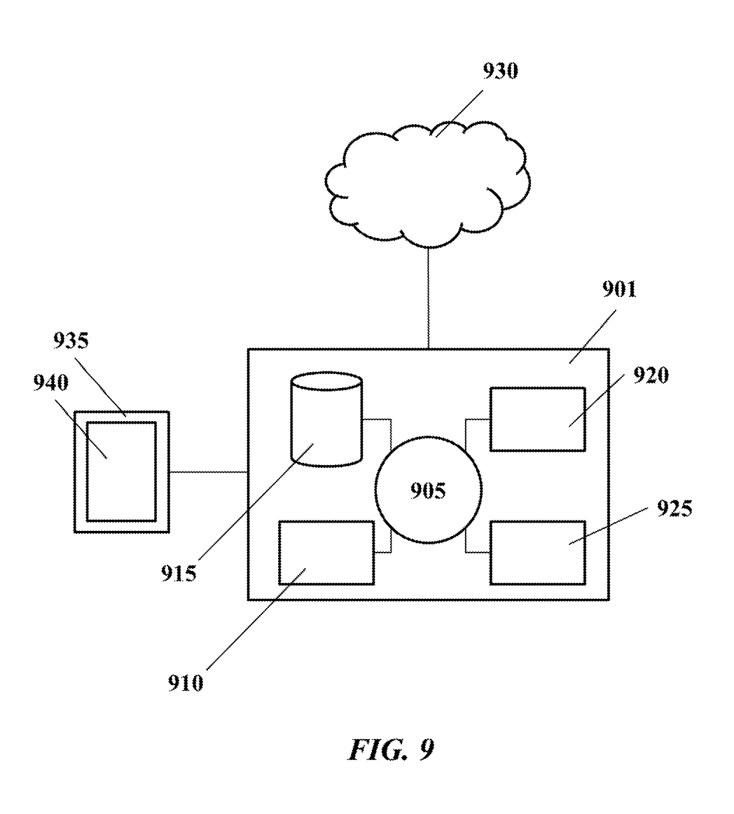

The present disclosure provides computer control systems that are programmed to implement methods of the disclosure. FIG. 9 shows a computer system 901 that is programmed or otherwise configured for sample processing and analysis, such as droplet generation and nucleic acid amplification and detection. The computer system 901 can regulate various aspects of methods and systems of the present disclosure.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet. The network 930 in some cases is a telecommunication and/or data network. The network 930 can include one or more computer servers, which can enable distributed computing, such as cloud computing. The network 930, in some cases with the aid of the computer system 901, can implement a peer-to-peer network, which may enable devices coupled to the computer system 901 to behave as a client or a server.

The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 901 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine executable or machine readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processor adapted to execute the code, or can be compiled during runtime. The code can be supplied in a programming language that can be selected to enable the code to execute in a pre-compiled or as-compiled fashion.

In another aspect, the present disclosure provides a non-transitory computer-readable medium comprising machine executable code that, upon execution by one or more computer processors, implements a method for conducting a chemical or biological reaction on a biological sample. The method may comprise depositing a solution comprising the biological sample in a sample holder, and the sample holder may retain the solution during the chemical or biological reaction. The sample holder may be disposed adjacent to a plurality of thermal zones comprising at least a first thermal zone and a second thermal zone. The second thermal zone may be angularly separated from the first thermal zone along an axis of rotation of (1) the sample holder or (2) the plurality of thermal zones. The method may further comprise alternately and sequentially positioning the solution in each of the plurality of thermal zones through rotation of the sample holder or the plurality of thermal zones, to conduct the chemical or biological reaction on the biological sample. In the first thermal zone, the solution may be subjected to heating or cooling at a first temperature profile, and in the second thermal zone, the solution may be subjected to heating or cooling at a second temperature profile that is different than the first temperature profile.

In another aspect, the present disclosure provides a non-transitory computer-readable medium comprising machine executable code that, upon execution by one or more computer processors, implements a method for generating at least one droplet comprising a biological sample for use in a chemical or biological reaction. The method may comprise activating an apparatus comprising (1) a first chamber comprising a first fluid volume and at least one first fluid flow port that is in fluid communication with the first fluid volume, wherein the first fluid volume comprises an aqueous solution comprising the biological sample for use in the chemical or biological reaction; and (2) a second chamber comprising a second fluid volume and at least one second fluid flow port that is in fluid communication with the second fluid volume, wherein the second chamber at least partially circumscribes the first chamber, wherein the second fluid volume retains a continuous fluid that is immiscible with the aqueous solution, and wherein the second chamber is rotatable with respect to the first chamber, or vice versa. The method may further comprise rotating the first chamber or the second chamber to bring the first fluid flow port in alignment with the second fluid flow port to subject the aqueous solution comprising the biological sample to flow from the first fluid volume to the second fluid volume to generate the at least one droplet upon the aqueous solution contacting the continuous fluid, which at least one droplet comprises the biological sample or a portion thereof.

In another aspect, the present disclosure provides a non-transitory computer-readable medium comprising machine executable code that, upon execution by one or more computer processors, implements a method for cooling a solution comprising a biological sample (e.g., nucleic acid sample) during a chemical or biological reaction (e.g., nucleic acid amplification reaction). The method may comprise activating a heat exchange apparatus comprising (1) a first chamber comprising a heat transfer material having a phase transition temperature in a range of about −100° C. to 50° C.; and (2) a second chamber comprising a substrate having a heat transfer surface. In some embodiments, the first chamber comprises a heat transfer material having a phase transition temperature of at least about −120° C., −110° C., −100° C., −95° C., −90° C., −85° C., −80° C., −75° C., −70° C., −65° C., −60° C., −55° C., −50° C., to about 0° C., 10° C., 20° C., 30° C., 40° C., or 50° C., such as, e.g., about −120° C. to 20° C., −110° C. to 30° C., −100° C. to 50° C., −100° C. to 60° C., −100° C. to 70° C., −95° C. to 55° C., −90° C. to 50° C., etc. The second chamber may be fluidically isolated from the first chamber, and the heat transfer surface may be in thermal communication with a solution comprising the biological sample during the chemical or biological reaction. The method may further comprise bringing the second chamber in fluid communication with the first chamber in accordance with a timing that at least partially depends upon a duration of the chemical or biological reaction. When the second chamber is in fluid communication with the first chamber, the heat transfer material may undergo a phase transition that draws thermal energy from the substrate along the heat transfer surface to subject the solution to cooling.

In another aspect, the present disclosure provides a non-transitory computer-readable medium comprising machine executable code that, upon execution by one or more computer processors, implements a method for cooling a solution comprising a biological sample (e.g., nucleic acid sample) during a chemical or biological reaction (e.g., nucleic acid amplification reaction). The method may comprise activating a heat exchange apparatus comprising: (1) a first chamber comprising a heat transfer material; (2) a second chamber comprising a substrate having a heat transfer surface, wherein the second chamber is fluidically isolated from the first chamber, and wherein the heat transfer surface is in thermal communication with the solution comprising the biological sample during the chemical or biological reaction; and (3) a seal between the first chamber and the second chamber, which seal (i) isolates the second chamber from the first chamber when in a closed configuration, and (ii) brings the second chamber in fluid communication with the first chamber when in an open configuration. The method may further comprise actuating the seal from the closed configuration to the open configuration to bring the first chamber in fluid communication with the second chamber. In the open configuration, the heat transfer material may undergo a phase transition that draws thermal energy from the substrate along the heat transfer surface to subject the solution to cooling.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that comprises a user interface (UI) 940 for providing, for example, nucleic acid sequence information. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 905. The algorithm can, for example, regulate systems or implement methods provided herein.

Devices, systems and methods of the present disclosure may be combined with other devices, systems or methods, such as those described in PCT/CN14/094914 and PCT/CN14/078022, each of which is entirely incorporated herein by reference.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-204. (canceled)

205. A system for conducting a chemical or biological reaction on a biological sample, comprising:
a sample holder that receives a solution comprising said biological sample, wherein said sample holder retains said solution during said chemical or biological reaction;
a plurality of thermal zones comprising at least a first thermal zone and a second thermal zone adjacent to said sample holder, wherein said second thermal zone is angularly separated from said first thermal zone along an axis of rotation of (1) said sample holder or (2) said plurality of thermal zones; and
a controller that alternately and sequentially positions said solution in each of said plurality of thermal zones through rotation of said sample holder or said plurality of thermal zones, to conduct said chemical or biological reaction, wherein (i) in said first thermal zone said solution is subjected to heating or cooling at a first temperature profile, and (ii) in said second thermal zone said solution is subjected to heating or cooling at a second temperature profile that is different than said first temperature profile.

206. The system of claim 205, wherein said first thermal zone comprises a first heating or cooling unit that subjects said solution to heating or cooling at said first temperature profile, and wherein said second thermal zone comprises a second heating or cooling unit that subjects said solution to heating or cooling at said second temperature profile.

207. The system of claim 205, wherein said first thermal zone and said second thermal zone are included on a support.

208. The system of claim 207, wherein said support is rotatable with respect to said sample holder.

209. The system of claim 205, wherein in said first thermal zone said solution undergoes heating and in said second thermal zone said solution undergoes cooling, or vice versa.

210. The system of claim 205, wherein said plurality of thermal zones further comprises a third thermal zone adjacent to said sample holder, wherein said third thermal zone is different than said first thermal zone and said second thermal zone, and wherein in said third thermal zone said solution is subjected to heating or cooling at a third temperature profile.

211. The system of claim 210, wherein said third temperature profile is different than said first temperature profile and said second temperature profile.

212. The system of claim 205, wherein said first temperature profile comprises a first target temperature, and wherein said second temperature profile comprises a second target temperature that is different than said first target temperature.

213. The system of claim 205, wherein said solution comprises reagents necessary for said chemical or biological reaction, and wherein said chemical or biological reaction is nucleic acid amplification, and wherein said reagents include one or more primers and polymerizing enzymes.

214. The system of claim 205, wherein said sample holder is rotatable with respect to said first thermal zone and said second thermal zone.

215. The system of claim 205, wherein said first thermal zone and said second thermal zone are rotatable with respect to said solution.

216. The system of claim 205, further comprising a detector adjacent to said sample holder, wherein said detector detects a signal from said solution that is indicative of said chemical or biological reaction or a product of said chemical or biological reaction on said biological sample.

217. The system of claim 216, wherein said detector is angularly separated from said first thermal zone and said second thermal zone along said axis of rotation.

218. A method for conducting a chemical or biological reaction on a biological sample, comprising:
(a) depositing a solution comprising said biological sample in a sample holder, wherein said sample holder retains said solution during said chemical or biological reaction, wherein said sample holder is disposed adjacent to a plurality of thermal zones comprising at least a first thermal zone and a second thermal zone, wherein said second thermal zone is angularly separated from said first thermal zone along an axis of rotation of (1) said sample holder or (2) said plurality of thermal zones; and
(b) alternately and sequentially positioning said solution in each of said plurality of thermal zones through rotation of said sample holder or said plurality of thermal zones, to conduct said chemical or biological reaction on said biological sample, wherein (i) in said first thermal zone said solution is subjected to heating or cooling at a first temperature profile, and (ii) in said second thermal zone said solution is subjected to heating or cooling at a second temperature profile that is different than said first temperature profile.

219. The method of claim 218, wherein (b) comprises positioning said solution in said first thermal zone and subsequently positioning said solution in said second thermal zone.

220. The method of claim 218, wherein (b) comprises positioning said solution in said first thermal zone and subsequently positioning said solution in a third thermal zone of said plurality of thermal zones that is different than said second thermal zone.

221. The method of claim 218, wherein in said first thermal zone said solution undergoes cooling and in said second thermal zone said solution undergoes heating, or vice versa.

222. The method of claim 218, wherein said first temperature profile comprises a first target temperature, and wherein said second temperature profile comprises a second target temperature that is different than said first target temperature.

223. The method of claim 218, wherein said sample holder is rotatable with respect to said first thermal zone and said second thermal zone.

224. The method of claim 218, wherein said first thermal zone and said second thermal zone are rotatable with respect to said solution.
